(19) United States
(12) Patent Application Publication
(10) Pub. No.: US 2025/0229089 A1
(43) Pub. Date: Jul. 17, 2025

(54) WCD WITH FIXED RATE DEMAND PACING AND DEMAND PACING WITH INTRINSIC SEARCH

(71) Applicant: West Affum Holdings DAC, Dublin (IE)

(72) Inventors: Pamela F. Breske, Newcastle, WA (US); David P. Finch, Bothell, WA (US)

(73) Assignee: West Affum Holdings DAC, Dublin (IE)

(21) Appl. No.: 19/019,018

(22) Filed: Jan. 13, 2025

Related U.S. Application Data
(60) Provisional application No. 63/621,075, filed on Jan. 15, 2024.

(51) Int. Cl. A61N 1/365; A61N 1/362

(52) U.S. Cl. CPC A61N 1/365; A61N 1/3625

(57) ABSTRACT

A medical system including a support structure to be worn by a patient, a plurality of ECG electrodes to sense ECG signals of the patient, at least one therapy electrode to deliver pacing pulses, and a processor in communication with the plurality of ECG electrodes and the at least one therapy electrode. The processor operates the medical system according to a demand pacing function, including a fixed pacing demand mode and an intrinsic search pacing demand mode. In the intrinsic search pacing demand mode, the processor operates the demand pacing function at a maximum pacing rate for a first time interval, followed by a minimum pacing rate during an intrinsic search duration, and evaluates heart activity via ECG signals to determine if it exceeds a threshold. In the fixed pacing demand mode, the processor operates the demand pacing function at a fixed pacing rate.

### CROSS-REFERENCE TO RELATED APPLICATIONS

This application claims priority to and the benefit of the provisional patent application No. 63/621,075 titled “WCD WITH FIXED RATE DEMAND PACING AND DEMAND PACING WITH INTRINSIC SEARCH”, filed in the United States Patent and Trademark Office on Jan. 15, 2024. The specification of the above referenced patent application is incorporated herein by reference in its entirety.

### TECHNICAL FIELD

The present technology relates to the field of wearable medical systems and more particularly, but not by way of limiting, the present technology relates to a medical system that is capable of providing demand pacing to a patient, involving electrocardiogram (ECG) sensing, rate control, and inhibition of pacing pulse delivery based on pre-defined condition(s).

### BACKGROUND

An exponential increase in sudden cardiac deaths (SCD), sudden cardiac arrests (SCA), and other cardiac related ailments is a significant global health concern. Patients having medical history related to cardiac events or who are at risk of SCA need to be monitored for any potential arrhythmias or emergencies that demand urgent attention and care. One of the solutions include a wearable cardioverter defibrillator (WCD), which is a non-invasive external device worn by patients who are at risk of potential life-threating situations. A WCD typically includes a built-in defibrillator that provides electric current (or shock) to restore a normal rhythm of the heart.

When a patient wears the WCD, one or more ECG signals of the patient are monitored to detect any abnormal behavior. The defibrillator may be primed to administer a suitable electric shock via the patient's body that shocks the heart if a potentially fatal arrhythmia is identified. WCDs can provide therapy for ventricular tachycardia (VT) or ventricular fibrillation (VF). However, currently known WCDs do not provide management and delivery of transcutaneous pacing therapy for drastic situations where individuals are prone to bradycardia or asystole.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure relates to a medical system that operates according to a demand pacing function. In one aspect of the present disclosure, the medical system includes a support structure configured to be worn by a patient, a plurality of ECG electrodes to sense one or more ECG signals of the patient, at least one therapy electrode, and a processor. The at least one therapy electrode, engaged to the support structure and in communication with an energy source, is configured to deliver one or more pacing pulses to the patient. The at least one therapy electrode may be positioned external to the patient and the one or more pacing pulses may be transcutaneous pacing pulses. The processor is in communication with the plurality of ECG electrodes and the at least one therapy electrode. The processor is configured to operate the medical system according to the demand pacing function, which is configured to initiate delivery of the zero or more pacing pulses. The demand pacing function comprises a fixed pacing demand mode and an intrinsic search pacing demand mode.

In the intrinsic search pacing demand mode, the processor is configured to cause the demand pacing function to operate at a maximum pacing rate for a first time interval. In an example, the maximum pacing rate is 80 beats per minute (bpm). Further, the processor is configured to decrease the pacing rate from the maximum pacing rate to a minimum pacing rate during a decreasing pacing rate period, which may be referred to as a second time interval hereinafter. Subsequently, the processor is configured to cause the demand pacing function to operate at the minimum pacing rate for an intrinsic search duration, which may be referred to as a third time interval hereinafter. Furthermore, the processor is configured to determine whether a heart activity of the patient exceeds a threshold during the intrinsic search duration via the one or more ECG signals. The one or more transcutaneous pacing pulses are delivered to the patient unless the patient's intrinsic heart rate is higher than the minimum pacing rate as higher intrinsic heart rates will inhibit the delivery of the one or more transcutaneous pacing pulses.

The heart activity of the patient includes zero or more intrinsic beats and/or zero or more paced beats during the intrinsic search duration. The processor is configured to determine a number of intrinsic beats that occurred during the intrinsic search duration. The heart activity of the patient exceeds the threshold when the number of intrinsic beats meets or exceeds a predetermined percentage of a total number of beats during the intrinsic search duration. The processor is configured to continue causing the demand pacing function to operate at the minimum pacing rate when the heart activity of the patient exceeds the threshold. The processor is further configured to cause the demand pacing function to operate at the maximum pacing rate for the first time interval when the heart activity of the patient does not exceed the threshold. In particular, the processor is configured to increase the pacing rate from the minimum pacing rate to the maximum pacing rate during an increasing pacing rate period, hereinafter referred to as fourth time interval, when the heart activity of the patient does not exceed the threshold. In addition, the processor is further configured to cause the demand pacing function to operate at a fixed pacing rate in the fixed pacing demand mode. During the fixed pacing demand mode, the above-mentioned maximum pacing rate and the minimum pacing rate may be equal to provide the fixed pacing rate.

### BRIEF DESCRIPTION OF THE DRAWINGS

The above-mentioned implementations are further described herein with reference to the accompanying figures. It should be noted that the description and figures relate to exemplary implementations and should not be construed as a limitation to the present disclosure. It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

FIG. 1 illustrates an example of a medical system worn by a patient, according to an embodiment of the present disclosure.

FIG. 2 illustrates a conceptual diagram with multiple electrodes of the medical system, according to an embodiment of the present disclosure.

FIG. 3 illustrates a block diagram of an external pacer and defibrillator (EPD), according to an embodiment of the present disclosure.

FIG. 4A illustrates a first pacing scenario for intrinsic search demand pacing depicting the occurrence of various transitions during the intrinsic search demand pacing, according to an embodiment of the present disclosure.

FIG. 4B illustrates a second pacing scenario for intrinsic search demand pacing depicting the occurrence of various transitions during the intrinsic search demand pacing, according to an embodiment of the present disclosure.

FIG. 5 illustrates a pacing pulse waveform for pacing, according to another embodiment of the present disclosure.

FIG. 6 illustrates a flowchart for fixed rate demand pacing and intrinsic search demand pacing, according to an embodiment of the present disclosure.

FIG. 7 illustrates an example method for managing a demand pacing function in a medical system, according to an embodiment of the present disclosure.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures or methods associated with the wearable medical system have not been shown or described in detail to avoid unnecessarily obscuring descriptions of the embodiments.

Unless the context indicates otherwise, throughout the specification and claims which follow, the word “comprise” and variations thereof, such as, “comprises” and “comprising” are to be construed in an open, inclusive sense that is as “including, but not limited to.” Further, the terms “first,” “second,” and similar indicators of the sequence are to be construed as interchangeable unless the context clearly dictates otherwise.

Reference throughout this specification to “one aspect” or “an aspect” means that a particular feature, structure, or characteristic described in connection with an embodiment is included in at least one aspect. Thus, the appearances of the phrases “in one aspect” or “in an aspect” in various places throughout this specification are not necessarily all referring to the same embodiment. Furthermore, the particular features, structures, or characteristics may be combined in any suitable manner in one or more aspects.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

Certain terms and phrases have been used throughout the disclosure and will have the following meanings in the context of the ongoing disclosure:

The term “pacing” refers to a method of applying electrical stimuli (or pacing pulses) to the heart of a patient, to control or pace the heart. Specifically, in pacing, low voltage electrical pulses are applied to the heart with the intent of causing contraction of the heart muscle that is between pacing electrodes. Each contraction causes the blood to flow out of the heart, providing potentially life-sustaining circulation to the rest of the body.

The term “demand pacing” refers to a mode of operation used by medical devices or systems to adjust the delivery of pacing pulses based on patient's needs. In demand pacing, the medical device (e.g., pacemaker) synchronizes with the heart's natural rhythm and only delivers pacing pulses when it detects that the patient's heart is not beating at a rate that exceeds the pacing rate of the demand pacemaker. Demand pacing typically includes ECG sensing to monitor the heart's natural rhythm, and inhibition of pacing pulse delivery based on pre-defined condition(s), such as, when intrinsic heart rate of the patient is higher than the pacing rate of the pacemaker.

The term “transcutaneous pacing” (TCP) refers to a non-invasive external pacing technique that is used to regulate a patient's heart rate by delivering electrical impulses to the heart through the skin. Within the context of the disclosure, TCP may be provided by placing external electrodes (pads) on the skin over the chest, typically one on the front (anterior) and one on the back (posterior) of the patient. However, any combination of small or large electrodes (e.g., pad to ECG sensor, or pad to pad) and/or different pad placements (e.g., anterior-lateral) may be utilized for providing TCP to the patient without limitation and other implementations may be executed without departing from the scope of the present disclosure. In the case of TCP, individual pacing pulses may not capture sufficient heart muscle to cause a contraction, so higher maximum pacing rates may need to be utilized than usual for intracardiac pacing. For example, at least 50 percent capture achieved by a higher TCP rate may lead to the patient having a life-sustaining rate of contraction. It should be noted that pacing, within the context of the disclosure, refers to external transcutaneous pacing unless specified otherwise in the present disclosure.

The term “intrinsic activity of the heart” refers to the natural and inherent ability of the heart to initiate electrical impulses under normal conditions and generate the heartbeat without external stimulation or intervention. The terms “intrinsic heart activity,” “intrinsic activity,” or “heart activity,” may be interchangeably used throughout the present disclosure.

The term “intrinsic heart rate” refers to the rate at which the heart would beat if there were no external influences, such as sympathetic or parasympathetic nervous systems. Intrinsic heart rate is typically around 60-100 bpm for an adult. The terms “intrinsic heart rate” or “heart rate” may be used interchangeably throughout the present disclosure.

Pacing regulates the heart rate and maintains rhythm in individuals or patients with certain cardiac conditions. However, in response to detecting arrhythmia (e.g., bradycardia) or a critical cardiac condition (e.g., new onset asystole), pacing may be provided at a higher rate that may prevent intrinsic heartbeats from occurring or reduce the occurrence of intrinsic heartbeats. Intrinsic heartbeats are believed to be better for the heart as they are natural electrical impulses generated by heart's conduction system and correspond to the heart's natural rhythm. Therefore, prevention or reduced occurrence of intrinsic heartbeats is generally undesirable during pacing.

Embodiments of the present disclosure describe methods and systems for managing the rate of a demand pacemaker to provide more opportunities for the patient's heart to generate intrinsic heartbeats. Specifically, some embodiments relate to a medical system (e.g., WCD) that can be configured to provide demand pacing to the patient. The medical system provides a demand pacing function that operates in either a fixed pacing demand mode or an intrinsic search pacing demand mode, based on a setting entered into the medical system. In either mode, the medical system only delivers pulses only when needed, i.e., when the intrinsic heart rate of the patient is less than the set pacing rate of the pacemaker. In various embodiments described herein, the medical system operating the demand pacing function includes using one or more ECG signals of the patient to sense intrinsic cardiac activity, a pacemaker with an ability to deliver pacing pulses at a configurable pacing rate, and an ability to inhibit delivery of pacing pulses when the intrinsic heart rate of the patient exceeds the pacemaker's pacing rate.

In embodiments pertaining to intrinsic search demand pacing, the pacing rate may oscillate between a maximum pacing rate and a minimum pacing rate. The intrinsic search demand pacing starts at the maximum pacing rate, which may be programmable. The pacing rate stays at the maximum pacing rate for a first time interval. Subsequently, the pacing rate decreases from the maximum pacing rate to the minimum pacing rate during a second time interval while attempting to detect intrinsic activity. Once the intrinsic search demand pacing is operating at the minimum pacing rate, the patient is monitored for intrinsic heartbeats. The pacing rate stays at the minimum pacing rate for a third time interval as long as the intrinsic heart rate of the patient exceeds the predetermined threshold. It should be noted that R-R intervals may change from beat to beat i.e., the timing between consecutive heartbeats of the patient 102 may vary. Therefore, in an exemplary embodiment, one or more pacing pulses may be delivered during the intrinsic search duration and still exceed the threshold, depending on the variability of the R-R intervals. The variation in the timing between consecutive heartbeats allows the medical system to adapt dynamically to the heart's natural rhythm while still providing demand pacing when necessary. In an example, if the minimum pacing rate is set at 60 bpm and the intrinsic heart rate of the patient is 70 bpm, then no pacing pulses will be delivered as the intrinsic heart rate of the patient exceeds the set minimum pacing rate. If the intrinsic heart rate of the patient is below the minimum pacing rate (for example, the intrinsic heart rate of the patient is 50 bpm while the set minimum pacing rate is 60 bpm), then the medical system will deliver the transcutaneous pacing pulses, and the intrinsic heart activity of the patient will not meet the predetermined threshold. Subsequently, the pacing rate is increased over a fourth time interval to the maximum pacing rate, which may be programmable as well. Once the maximum pacing rate is reached, the medical system repeats the process for intrinsic search demand pacing. Each of the embodiments are further described in detail in conjunction with various figures.

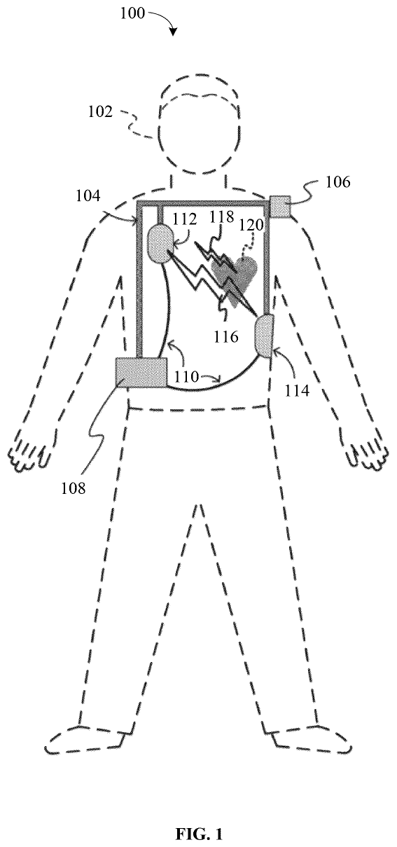

FIG. 1 illustrates an example of a medical system 100 worn by a patient 102, according to an embodiment of the present disclosure. The patient 102 may also be referred to as a person 102 and/or a wearer 102, since the patient 102 is wearing components of the medical system 100, or as a user 102 using the medical system 100. The patient 102 could be ambulatory, i.e., while wearing the medical system 100, the patient 102 can walk around and is not necessarily bed-ridden. While the patient 102 may also be considered to be a “user” of the medical system 100, this definition is not exclusive to the patient 102. For instance, the user 102 of the medical system 100 may also be a clinician such as a doctor, nurse, emergency medical technician (EMT), or other similarly tasked individual or group of individuals. In some cases, the user 102 may even be a bystander. The particular context of these and other related terms within this description should be interpreted accordingly.

In an example, the medical system 100 may be referred to as a wearable medical system (WMS). In some embodiments, the medical system 100 may be a wearable cardioverter defibrillator (WCD). The medical system 100 at least includes one or more components such as a support structure 104, an outside monitoring device 106, an external pacer and defibrillator (EPD) 108, and electrode leads 110 that allow coupling of defibrillation electrodes 112 and 114 to the EPD 108.

The support structure 104 may be configured to be worn by the patient 102 for at least several hours per day, during the night, one or more days, and/or one or more months. The support structure 104 may be implemented in many different ways. For example, the support structure 104 may be implemented in a single component or a combination of multiple components. In some embodiments, the support structure 104 may include a vest, a half-vest, a garment, or the like such that the support structure 104 may be worn similarly to analogous articles of clothing. In some embodiments, the support structure 104 may include a harness, one or more belts or straps, and the like. In some embodiments, the support structure 104 may be worn by the patient 102 around the torso, hips, over the shoulder, and the like. In some embodiments, the support structure 104 includes a container or housing that may be waterproof. Further, the support structure 104, in some embodiments, may be worn by being attached to the patient's body by an adhesive material, for example as shown and described in U.S. Pat. No. 8,024,037. The support structure 104 may be implemented as a support structure described in U.S. Patent Publication No. 2017/0056682 A1, which is incorporated herein by reference. The person skilled in the art will recognize that the components of the medical system 100 may reside in the housing of the support structure 104 instead of being attached externally to the support structure 104, for example as described in the aforementioned '682 document. It shall be understood that the support structure 104 is shown generically in FIG. 1 and merely illustrates concepts about the support structure 104. FIG. 1 is not to be construed as limiting with respect to either a manner in which the support structure 104 is implemented or how the support structure 104 is worn. Also, the support structure 104 may be implemented in various other examples.

According to some embodiments, the medical system 100 may obtain data from the patient 102 which is referred to as patient data. For collecting the patient data, the medical system 100 may, in some embodiments, include at least the outside monitoring device 106, also referred to as a device 106 hereinafter. The device 106 may be provided as a standalone device, for example, external to the EPD 108. The device 106 may be configured to sense or monitor one or more local parameters. The one or more local parameters may be one or more parameters of the patient 102, one or more parameters of the medical system 100, or one or more parameters of the environment, without limitation.

The device 106 may include one or more sensors for obtaining the one or more parameters. Each of the one or more sensors may be configured to sense the one or more parameters of the patient 102, the medical system 100, and/or the environment. Each of the one or more sensors are further configured to render an input responsive to the sensed one or more parameters. In some embodiments, the rendered input is quantitative, such as values of a sensed parameter. In some embodiments, the input is qualitative, such as indicating whether one or more thresholds are crossed, and the like. In some embodiments, the rendered inputs about the patient 102 are also called physiological inputs or patient inputs. In some embodiments, a sensor may be construed more broadly, as encompassing more than one individual sensor.

In some embodiments, the device 106 may be physically coupled to the support structure 104. Additionally, the device 106 may be communicatively coupled with other components that are coupled to the support structure 104. The communication between the device 106 and the other components may be implemented by a communication module, as will be deemed applicable by a person skilled in the art in view of the present disclosure.

The EPD 108 is also referred to as a pacer 108 or as a main electronics module 108. A component of the EPD 108 may be configured to store electrical charge. Other components may cause at least some of the stored electrical charge to be discharged via the defibrillation electrodes 112 and 114, for delivering electrical pulses to the patient 102. The EPD 108 may initiate defibrillation, hold-off defibrillation, or initiate pacing, based on a combination of a variety of inputs, with the ECG signal merely being one of the varieties of inputs.

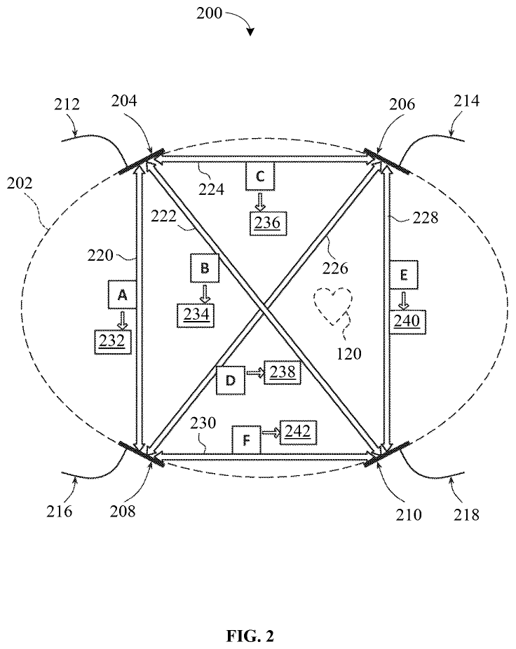

The defibrillation electrodes 112 and 114 are also referred to as electrotherapy electrodes 112 and 114, therapy electrodes 112 and 114, or pacing electrodes 112 and 114. Although the defibrillation electrodes 112 and 114 are depicted as plurality of therapy electrodes, a single therapy electrode or more than two therapy electrodes may be utilized for same functionality without limitation. Accordingly, a single therapy electrode or plurality of therapy electrodes may be utilized and referred to as at least one therapy electrode 112, 114 in the present disclosure. The defibrillation electrodes 112 and 114 may be configured to be positioned on the body of the patient 102 in a number of ways. In embodiments, the at least one therapy electrode 112, 114 may be positioned external to the patient 102. For instance, the EPD 108 and one or more of the defibrillation electrodes 112 and 114 may be engaged with the support structure 104, directly or indirectly. For example, in some embodiments, one or more of the defibrillation electrodes 112 and 114 may be engaged with the support structure 104 by being integrated with or attached to a housing of the support structure 104, disposed in pocket(s) of the support structure 104, and/or coupled to the support structure 104 using fasteners such as snaps, clips, hook-loop fasteners, and the like. In an example, the support structure 104 may be configured to be worn by the ambulatory patient 102 to maintain at least one of the defibrillation electrodes 112 and 114 on the body of the patient 102, while the patient 102 is moving around. The one or more of the defibrillation electrodes 112 and 114 may be thus maintained on the body of the patient 102 in electrical contact of the skin of the patient 102 by the support structure 104, such that the defibrillation electrodes 112 and 114 are pressed against the skin directly or through conductive portions of the support structure 104 (e.g., a garment), and the like, of the patient 102. Further, the defibrillation electrodes 112 and 114 may also sense ECG signals of the patient 102 and be utilized as ECG electrodes or sensing electrodes, which will be described later with respect to FIG. 2.

In some embodiments, the defibrillation electrodes 112 and 114 are not necessarily pressed against the skin but may become biased upon sensing a condition that may merit intervention by the medical system 100. Additionally, some of the components of the EPD 108 may be considered coupled to the support structure 104 directly, or indirectly via at least one of the defibrillation electrodes 112 and 114.

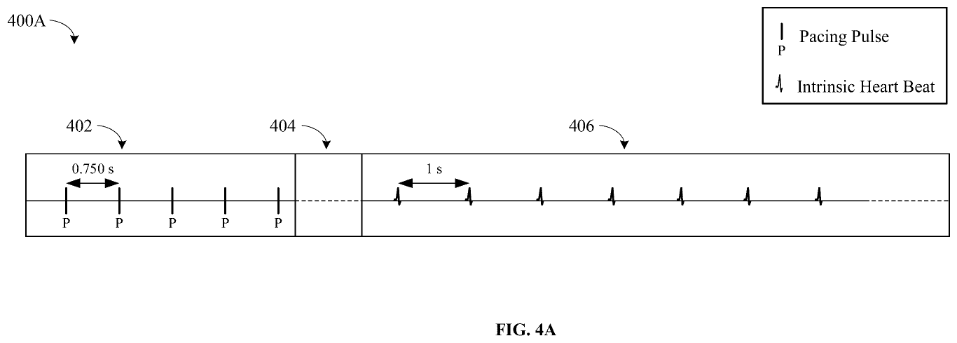

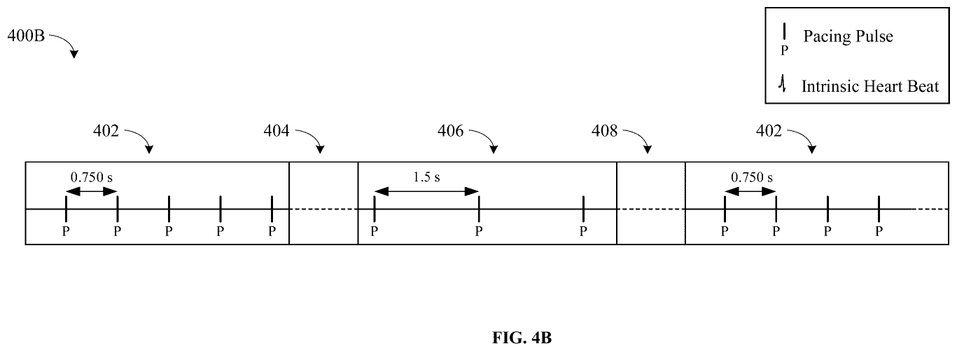

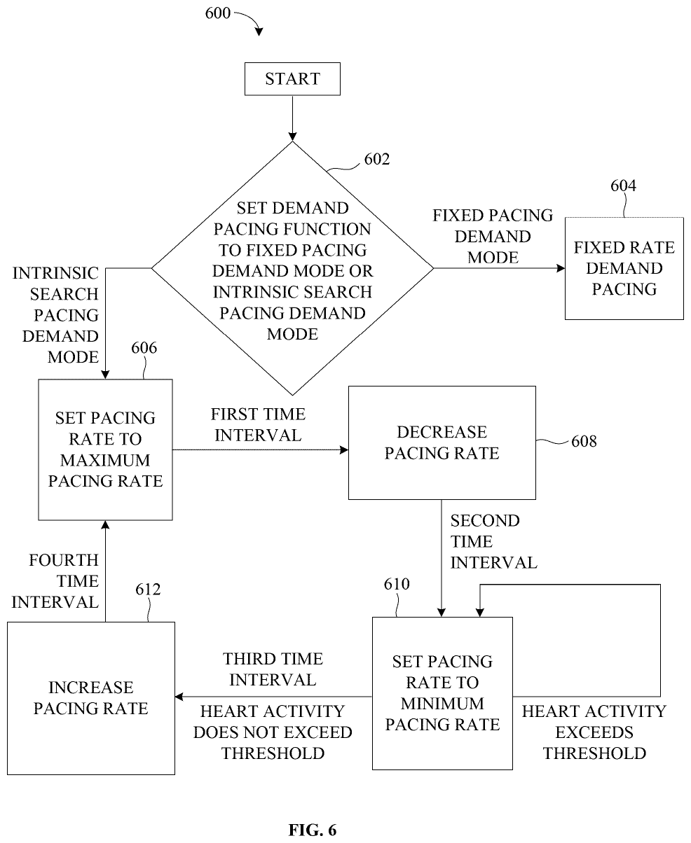

The electrical pulses may be categorized based on energy of the electrical pulses. The electrical pulses are categorized as defibrillation shock 116 and one or more pacing pulses 118 that are typically much lower in energy than the defibrillation shock 116. In embodiments, the one or more pacing pulses 118 are transcutaneous pacing pulses, and may be used interchangeably depending on the context in the present disclosure. The action of delivering the defibrillation shock 116 is also called shocking the patient 102, and the action of delivering the one or more transcutaneous pacing pulses 118 is called pacing. The one or more transcutaneous pacing pulses 118 are intended to pace the heart 120 if needed, and typically the one or more transcutaneous pacing pulses 118 are caused to be delivered in a periodic sequence by appropriately timed discharges. The defibrillation shock 116 is also referred to as cardioversion shock, therapy shock, or the like. The electrical pulses corresponding to the defibrillation shock 116 may also be referred to as defibrillation pulses 116. In accordance with the embodiments of the present disclosure, the EPD 108 may also include one or more modules to implement an intrinsic search pacing demand mode or fixed pacing demand mode for the patient 102. The intrinsic search pacing demand mode is described in detail in conjunction with FIG. 4A, FIG. 4B, and FIG. 6 later.

When the defibrillation electrodes 112 and 114 make good electrical contact with the body of the patient 102, the EPD 108 may administer one or more brief electric pulses to the body of the patient 102, such as the defibrillation shock 116 or the one or more transcutaneous pacing pulses 118 via the defibrillation electrodes 112 and 114. The administration of the defibrillation shock 116 or the one or more transcutaneous pacing pulses 118, based on corresponding requirement, is referred to as electrotherapy. The defibrillation shock 116 or the one or more transcutaneous pacing pulses 118 have attributes suitable for their purpose.

The defibrillation shock 116 is typically stronger than the one or more transcutaneous pacing pulses 118 such that the defibrillation shock 116 may have an energy of at least 100 Joules (J), for example, 200 J, 300 J, 360 J, and the like. The defibrillation shock 116 is intended to go through and reset the heart 120 of the patient 102 from a ventricular arrhythmia such as fibrillation or tachycardia back to a normal rhythm, in an effort to save the life of the patient 102.

The one or more transcutaneous pacing pulses 118 are not intended to be administered concurrently with the defibrillation shock 116. The one or more transcutaneous pacing pulses 118 are depicted to be smaller than the defibrillation shock 116 in FIG. 1 to reflect that the one or more transcutaneous pacing pulses 118 have less energy than the defibrillation shock 116. In an example, the one or more transcutaneous pacing pulses 118 may deliver current in the range of 100-200 milliamperes (mA) for a duration less than 50 milliseconds (ms). In a specific example, a 50 ms pacing pulse at 200 mA delivered into the patient 102 with a kiloohm resistance may be 2 J of energy, with typical pulses being less than 1 J.

In some embodiments, the one or more transcutaneous pacing pulses 118 are a discharge from at least the two electrodes, which are either the same defibrillation electrodes 112 and 114 used for the defibrillation shock 116, or different therapy electrodes (not shown). Further, during the application of external pacing, the medical system 100 is configured in some embodiments to alert bystanders or rescuers (e.g., medical personnel or emergency responders) and/or transmit notifications regarding the application of external pacing to remote people such as family members, the patient's doctor, and the like.

Intrinsic heartbeats are believed to be better for the patient's heart 120 than the one or more transcutaneous pacing pulses 118. In accordance with embodiments of the present disclosure, the medical system 100 may be operated according to a demand pacing function where the pacing performed may be fixed rate demand pacing, while in other embodiments the pacing may be intrinsic search demand pacing. The demand pacing function includes a fixed pacing demand mode and an intrinsic search pacing demand mode, based on a setting entered into the medical system 100 by the clinician or a technician (rather than the patient 102 or the medical system 100 autonomously changing the pacing). Precisely, in either the fixed pacing demand mode or the intrinsic search pacing demand mode, the medical system 100 delivers the one or more transcutaneous pacing pulses 118 only when the intrinsic heart rate of the patient 102 falls below a preset fixed rate of the EPD 108. However, if the heart 120 of the patient 102 beats at a rate higher than the preset fixed rate, the EPD 108 may not intervene.

When the clinician selects the fixed pacing demand mode, the medical system 100 is configured to operate the demand pacing function at a constant pacing rate. The constant pacing rate is maintained throughout the duration of a single demand pacing session. Further, when the clinician selects the intrinsic search pacing demand mode, the medical system 100 is configured to operate the demand pacing function at a maximum pacing rate for a first time interval. In embodiments, the first time interval may either be a fixed or a non-fixed time interval. This is followed by a gradual decrease of the pacing rate from the maximum pacing rate to a minimum pacing rate during a second time interval. The medical system 100 is further configured to operate the demand pacing function at the minimum pacing rate in order to search for intrinsic heart rate of the patient 102 for a third time interval. The third time interval may be fixed or non-fixed time interval. The intrinsic search demand pacing is a search, by gradually reducing the pacing rate for demand pacing, for natural electrical impulses that originate within the heart 120 and drive the rhythmic contractions. The gradual decrease of the pacing rate and an extended period of operating the medical system 100 at the minimum pacing rate, allows confirmation of the bradycardia still persistent in the patient 102 and then automatic return to the maximum pacing rate in a patient with a confirmed bradycardia. Embodiments of the intrinsic search demand pacing will be described in more detail below in conjunction with FIG. 4A, FIG. 4B, and FIG. 6. Since the intrinsic heartbeats are believed to be better for the patient's heart 120, so some embodiments of intrinsic search demand pacing can advantageously detect whether the patient 102 has intrinsic heartbeats and adjust the pacing so as to reduce the number of delivered external pacing pulses.

In some embodiments, the one or more components of the medical system 100 may be customized for the patient 102. The customization may include one or more aspects, such as providing the support structure 104 that is custom-fit for the body of the patient 102. Further, baseline physiological parameters of the patient 102 may be measured for various scenarios, such as when the patient 102 is lying down (in various orientations), sitting, standing, walking, running, and the like. The baseline physiological parameters may include heart rate of the patient 102, motion detector outputs, one for each scenario, and the like. Values of the measured baseline physiological parameters may be used to customize the medical system 100, to make accurate diagnoses for the patient 102. The customization of the medical system 100 allows other patients with bodies different from one another to use the medical system 100. Values of the measured baseline physiological parameters may be stored in a memory of the medical system 100, and so on. A programming interface, in some embodiments, receives the measured values of the baseline physiological parameters. The programming interface may provide an input related to the measured values of the baseline physiological parameters to the medical system 100 automatically, along with other data.

FIG. 2 illustrates a conceptual diagram 200 with multiple electrodes of the medical system 100, according to an embodiment of the present disclosure. FIG. 2 is described in conjunction with the previous figure. In some embodiments, the medical system 100 uses a multichannel ECG sensing system. The conceptual diagram 200 illustrates a section 202 of the patient 102 with multiple ECG electrodes 204, 206, 208, and 210 of the medical system 100 positioned around the heart 120 of the patient 102. The ECG electrodes 204, 206, 208, and 210 are also referred to as ECG sensing electrodes 204, 206, 208, and 210, and collectively referred to as ECG electrodes 204-210. The patient 102 is viewed from the top and the patient 102 is facing downwards. The section 202 is obtained when a plane intersects the patient 102 at the torso. The ECG electrodes 204-210 may be maintained on or surround the torso of the patient 102, and have respective wire leads 212, 214, 216, and 218. The ECG electrical potentials that may be measured at the ECG electrodes 204, 206, 208, and 210 have values E1, E2, E3, and E4, respectively.

Any pair of the ECG electrodes 204, 206, 208, and 210 defines a vector, along which an ECG signal may be sensed and/or measured. The ECG electrodes 204, 206, 208, and 210 pairwise define vectors 220, 222, 224, 226, 228, and 230, thereby illustrating a multi-vector embodiment. In some embodiments, although the ECG electrodes 204, 206, 208, and 210, and the vectors 220, 222, 224, 226, 228, and 230 are illustrated, other number of ECG electrodes and/or vectors may be implemented. In some embodiments, all the vectors 220, 222, 224, 226, 228, and 230 may not be considered. For example, the vectors 224 and 230 may be ignored since the vectors 224 and 230 least traverse the torso of patient 102 compared to other vectors such as the vectors 220, 222, 226, and 228.

It will be understood that the ECG electrodes 204, 206, 208, and 210 are illustrated to be shown on a same plane for simplicity of explanation. However, the ECG electrodes 204, 206, 208, and 210 may not necessarily exist on the same plane. Consequently, the vectors 220, 222, 224, 226, 228, and 230 may not necessarily exist on the same plane either. The vectors 220, 222, 224, 226, 228, and 230 define channels A, B, C, D, E, and F, respectively. ECG signals 232, 234, 236, 238, 240, and 242 may thus be sensed and/or measured from the channels A, B, C, D, E, and F, respectively, and particularly from the appropriate pairings of the wire leads 212, 214, 216, and 218 for each channel. The ECG signals 232, 234, 236, 238, 240, and 242, also collectively referred to as ECG signals 232-242, may or may not be sensed concurrently.

The above-mentioned formalism renders values of the ECG signals 232-242 that are sensed between pairs of the ECG electrodes 204, 206, 208, and 210 using the vectors 220, 222, 224, 226, 228, and 230. For example, the ECG signal 232 at channel A has a voltage E1−E2=E12. In some embodiments, a different formalism is utilized for deriving ECG signal values for each of the ECG electrodes 204, 206, 208, and 210 by itself, and at a corresponding location and not necessarily in a pair with another ECG electrode.

The different formalism includes considering a point at a virtual position (not shown) between the four ECG electrodes 204, 206, 208, and 210 within the torso of the patient 102. An average ECG voltage value (CM) may be ascribed to that point. The CM is derived from a statistic of voltages at the ECG electrodes 204, 206, 208, and 210. The virtual position continuously changes based on voltages of the ECG electrodes 204, 206, 208, and 210. However, an actual sensor for sensing the voltage at that point is ignored or not considered. Nevertheless, the different formalism further considers a virtual main central terminal (MCT) (not shown), which senses the CM. In the different formalism, therefore, the vectors 220, 222, 224, 226, 228, and 230 are considered from each of the ECG electrodes 204, 206, 208, and 210 to the MCT. Relative to the MCT, there may be four resulting vectors with values of the corresponding signals, which may be considered as: E1C=E1−CM, E2C=E2−CM, E3C=E3−CM, and E4C=E4−CM. In some embodiments, the vectors 220, 222, 224, 226, 228, and 230 are formed in software by selecting a pair of the signals and subtracting one from the other. For example, E1C−E2C=(E1−CM)−(E2−CM)=E1−E2+(CM−CM)=E1−E2=E12. Thus, by having the multiple channels A, B, C, D, E, and F, the medical system 100 may assess which one of the multiple channels A, B, C, D, E, and F provides the best ECG signal for intrinsic heartbeat analysis. Alternatively, instead of just one channel, the medical system 100 may determine to keep two or more, but not all, of the channels and use the corresponding ECG signals 232, 234, 236, 238, 240, and 242 for the intrinsic heartbeat analysis, for instance as described in U.S. Pat. No. 9,757,581, issued on Aug. 23, 2017.

In some embodiments, the medical system 100 may be implemented with multiple ECG electrodes, beyond the ECG electrodes 204-210 to generate multiple vectors (or channels) for monitoring the rhythm of the heart 120 of the patient 102. The medical system 100 continuously monitors the corresponding ECG signals 232-242 of the patient 102 to detect the cardiac rhythm disorders and may also monitor, in a further enhancement, activity of the patient 102 for noise detection.

The medical system 100 may also include the therapy electrodes 112 and 114, such as the defibrillation electrodes 112 and 114, for delivering the transcutaneous pacing pulses, such as the one or more transcutaneous pacing pulses 118, in response to detection of bradycardia or asystole. The use of multiple vectors may help in an improved and accurate intrinsic heartbeat analysis that includes detection of intrinsic beats in the received ECG signals 232-242.

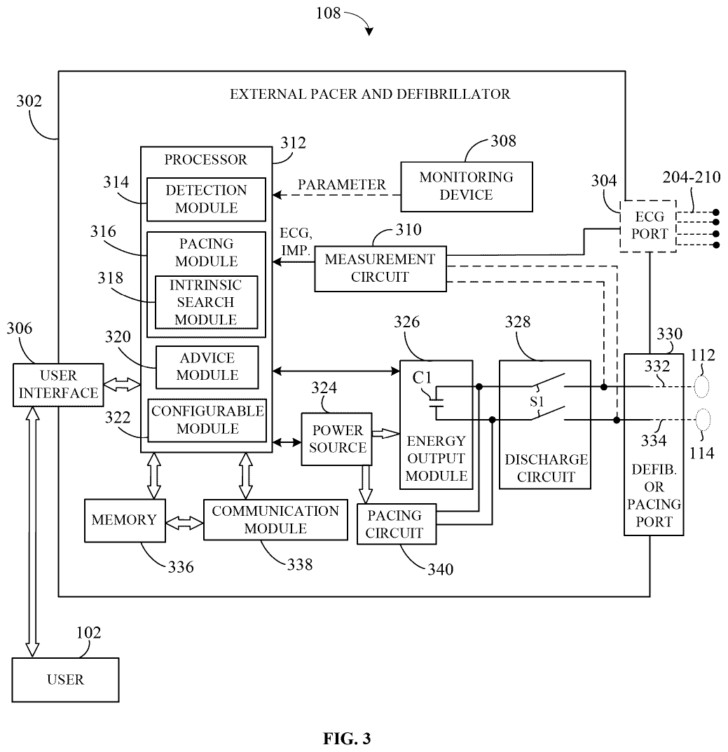

FIG. 3 illustrates a block diagram of an EPD 108 that is capable of providing the pacing to the patient 102, according to an embodiment of the present disclosure. FIG. 3 is described in conjunction with the previous figures. The EPD 108 is configured to provide intrinsic search demand pacing. In some embodiments, the EPD 108 is intended for the patient 102 who may be carrying the EPD 108 on their body, such as the ambulatory patient 102.

One or more components of the EPD 108 are stored in a housing 302, which may also be referred to as a casing 302. The EPD 108 at least includes components such as an ECG port 304, a user interface 306, a monitoring device 308, a measurement circuit 310, and a processor 312 including a plurality of modules such as a detection module 314, a pacing module 316 comprising an intrinsic search module 318, an advice module 320, and a configurable module 322. The EPD 108 further includes a power source 324, an energy output module 326, a discharge circuit 328, a defibrillation or pacing port 330 including nodes 332 and 334 and coupled to the defibrillation electrodes 112 and 114, a memory 336, a communication module 338, and a pacing circuit 340. The terms “external pacer and defibrillator (EPD)” and “medical system” are interchangeably used since the medical system 100 includes the EPD 108 unless the context clearly dictates otherwise.

The user interface 306 may include one or more output devices, which may be visual, audible, audio, or tactile, for communicating with the user 102 by outputting images, sounds, or vibrations. The communicated output perceivable by the patient 102 or the user 102 may also be called human-perceptible indications (HPIs). The HPIs may be used to alert the patient 102, provide sound alarms that may be intended also for bystanders, and the like. For example, an output device of the one or more output devices may be a light that may be turned on and off, a screen to display sensed, detected, and/or measured information by the medical system 100 and provide visual feedback to a rescuer, such as the user 102, for resuscitation attempts, and the like. Another output device of the one or more output devices may be a speaker, which may be configured to issue voice prompts, alerts, beeps, loud alarm sounds and/or words, and the like. The output provided by the one or more output devices may be communicated to the user 102, such as the bystander, when defibrillating or just pacing, and so on.

The user interface 306 may further include one or more input devices for receiving inputs from the user 102, such as the patient 102, the local trained caregiver, the bystander, and the like. In some embodiments, the user 102 may be a local rescuer at a scene, such as the bystander who might offer assistance, or a trained person. In some embodiments, the user 102 may be a remotely located trained caregiver in communication with the medical system 100. The one or more input devices may include various controls, such as push buttons, keyboards, touchscreens, one or more microphones, and the like.

One of the one or more input devices may be a cancel switch, also referred to as an “I am alive” switch or “live man” switch. Actuating the cancel switch, for example, may prevent the impending delivery of the defibrillation shock 116, or the one or more transcutaneous pacing pulses 118, to the patient 102. In some embodiments, the output device such as the speaker may be configured to output a warning prompt prior to the impending or planned defibrillation shock 116 or pacing sequence of the one or more transcutaneous pacing pulses 118 being caused to be delivered. The cancel switch is configured to be actuated by the patient 102 in response to the warning prompt.

The impending or planned defibrillation shock 116 or pacing sequence of the one or more transcutaneous pacing pulses 118 is caused to halt responsive to the actuation of the cancel switch after the warning prompt has been output. Operations of the processor 312 and methods may include causing the speaker to output the warning prompt and determining whether or not the cancel switch has been actuated after the warning prompt has been output. In some embodiments, the cancel switch may provide audio and/or haptic outputs as part of the alerts that the medical system 100 provides to the patient 102.

The ECG port 304, also referred to as a sensor port 304, is coupled to or adapted for plugging in one or more of the ECG electrodes 204-210. The ECG electrodes 204-210, in some embodiments, are resistive, DC-coupled ECG electrodes. The ECG electrodes 204-210, for example, may be connected continuously to the ECG port 304. In an example, an impedance of the ECG electrodes 204-210 may vary based on the patient 102, such as dryness or moisture of the skin, a manner in which the ECG electrodes 204-210 contact the skin, location of placement of the ECG electrodes 204-210, and the like.

The ECG electrodes 204-210 may sense an ECG signal, for example, a 12-lead signal. In some embodiments, the ECG electrodes 204-210 may sense a signal from a different number of leads, especially if the ECG electrodes 204-210 make good electrical contact with the body of the patient 102 and in particular with the skin of the patient 102. The ECG electrodes 204-210 may be attached to the inside of the support structure 104, for making good electrical contact with the patient 102. In some embodiments, the defibrillation electrodes 112 and 114 may be attached to the inside of the support structure 104. The ECG electrodes 204-210 continue to sense the ECG signal during the delivery of the defibrillation shock 116, or the one or more transcutaneous pacing pulses 118. Without departing from the scope of the present disclosure, a common set of electrodes may function as the ECG electrodes 204-210 as well as the defibrillation electrodes 112 and 114, and pacing may be performed using the common set of electrodes.

The monitoring device 308 of the EPD 108 is also referred to as an internal monitoring device 308 since the monitoring device 308 is incorporated within the housing 302. The monitoring device 308 may sense or monitor patient parameters such as physiological parameters of the patient 102, state parameters of the patient 102, system parameters, and/or environmental parameters, all of which may be referred to as patient data. In an example, the monitoring device 308 may include or may be coupled to one or more sensors. In some embodiments, the monitoring device 308 may be complementary or an alternative to the outside monitoring device 106. Allocating which patient parameters are to be monitored by the monitoring device 308 and the device 106 may be determined according to design considerations.

The physiological parameters of the patient 102, for example and without limitation, include one or more physiological parameters data that may assist the EPD 108 in detecting whether or not the patient 102 needs a shock, other intervention, or assistance. The physiological parameters may also, in an example, include physiological parameters data such as medical history of the patient 102, event history, and the like. The physiological parameters data may further include ECG, blood oxygen level, blood flow, blood pressure, blood perfusion, pulsatile change in light transmission or reflection properties of perfused tissue, heart sounds, heart wall motion, respiration-related information, breathing sounds, and pulse of the patient 102.

Accordingly, the monitoring device 308 and/or the outside monitoring device 106 may include one or more sensors configured to acquire patient physiological signals. In some embodiments, the one or more sensors may include the ECG electrodes 204-210 to detect or obtain the ECG signals 232-242, a perfusion sensor, a pulse oximeter, a device for detecting blood flow, for example, a Doppler device, and the like. In some embodiments, the one or more sensors may include a sensor for detecting blood pressure, for example, a cuff, an optical sensor, illumination detectors, and the one or more sensors perhaps working together with light sources for detecting a color change in a tissue. In some embodiments, the one or more sensors may include a motion sensor, a device that may detect the heart's wall motion or movement, a sound sensor, a device with a microphone, a SpO2 sensor, and the like. In view of the present disclosure, it will be appreciated that such sensors may help to detect the pulse of the patient 102 and may therefore also be called as pulse detection sensors, pulse sensors, or pulse rate sensors. In addition, a person skilled in the art may implement other ways of performing pulse detection.

In some embodiments, the monitoring device 308, the outside monitoring device 106, and/or the processor 312 may detect a trend in the monitored physiological parameters data of the patient 102. The trend may be detected by comparing values of parameters at different times over short and/or long terms. The physiological parameters whose detected trends may help a cardiac rehabilitation program include a) cardiac function, for example, ejection fraction, stroke volume, cardiac output, and the like; b) heart rate variability at rest or during exercise; c) heart rate profile during exercise and measurement of activity vigor, such as from the profile of an accelerometer signal and informed from adaptive rate pacemaker technology; d) heart rate trending; e) perfusions, such as from SpO2, CO₂, or other parameters such as those mentioned above; f) respiratory function, respiratory rate, and the like; g) motion, level of activity; and other similar parameters.

The detected trend may be stored and/or reported via one or more wired or wireless communication links, along with a warning if warranted to a physician monitoring progress or health status of the patient 102. The reported trends provide clarity and updated information corresponding to the patient 102, to the physician. The physician may gauge a condition that is either not improving or deteriorating based on the reported trends.

The state parameters may include recorded aspects of the patient 102, such as but not limited to the motion, posture, whether the patient 102 has spoken or communicated with a physician recently along with what has been spoken, and the like. In some embodiments, the state parameters may further include a history of the state parameters. In an example, the monitoring device 308 may include a location sensor such as a Global Positioning System (GPS) location sensor. The location sensor may detect the location of the patient 102, and speed may be detected as a rate of change of location over time.

In some embodiments, the monitoring device 308 may include the motion detectors that may be configured to detect a motion event and output a motion signal indicative of motion of the motion detectors, and thus the motion of the patient 102. The motion event may correspond to change in body position, sleep orientation, and the like, associated with the patient 102. The state parameters may assist in narrowing down the determination of whether SCA is indeed occurring. In some embodiments, the medical system 100 may include the motion detectors. The motion detectors may be implemented in many ways, for example, by using an accelerometer. The motion event may be defined as convenient, such as a change in motion from a baseline motion or rest, and the like. In an example, the motion detectors are implemented within the monitoring device 308. In response to the detected motion event, the motion detectors may render or generate a motion detection input that may be received by a subsequent device or functionality.

The system parameters of the medical system 100 may include system identification, battery status, system date and time, reports of self-testing, records of data entered, records of episodes and interventions, and the like. The environmental parameters may include ambient temperature and pressure. Moreover, a humidity sensor may provide information as to whether or not it is likely raining. The detected location of the patient 102 may also be considered as one of the environmental parameters. The patient's location may be presumed or considered, if the monitoring device 308 or the outside monitoring device 106 includes the GPS location sensor as mentioned above, and if the patient 102 is wearing the medical system 100.

The EPD 108 may also include the measurement circuit 310 that may be communicatively coupled to the sensors and the monitoring device 308, in some embodiments. The measurement circuit 310 may be configured to sense one or more electrical physiological signals of the patient 102 from the sensor port 304. In some embodiments, if the EPD 108 lacks the sensor port 304, the measurement circuit 310 may, in an example, obtain physiological signals through nodes 332 and 334 instead, when the defibrillation electrodes 112 and 114 are attached to the patient 102. The input to the measurement circuit 310 through the nodes 332 and 334 is an ECG signal that reflects the ECG measurement. The patient data, in an example, may be the ECG signals 232-242 that may be sensed as a voltage difference between the defibrillation electrodes 112 and 114. In addition, the patient parameters may include an impedance, which may be sensed between the defibrillation electrodes 112 and 114 and/or between the connections of the sensor port 304 considered pairwise.

Sensing the impedance may be useful for detecting, among other processes, whether the defibrillation electrodes 112 and 114 and/or the sensing electrodes 204-210 are making good electrical contact with the body of the patient 102. The patient's physiological signals may be sensed when available. The measurement circuit 310 may render or generate information about the patient's physiological signals as inputs, data, other signals, and the like. As such, the measurement circuit 310 may be configured to render the patient inputs responsive to the patient parameters sensed by the one or more sensors. In some embodiments, the measurement circuit 310 may be configured to render the patient inputs, such as values of the ECG signals 232, 234, 236, 238, 240, and 242, responsive to the ECG signals 232, 234, 236, 238, 240, and 242 sensed by the ECG electrodes 204-210. Information rendered by the measurement circuit 310 is an output, however, output information may be called an input because the information is received as an input by a subsequent device or functionality.

The EPD 108 also may include the processor 312 that may be implemented in different ways in various embodiments. The different ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination thereof, and the like. In some embodiments, the processor 312 may be implemented using multiple electronic devices distributed in various parts of the EPD 108.

The processor 312 may include, or have access to, a non-transitory storage medium, such as the memory 336 that, in some embodiments, is a non-volatile component for storage of machine-readable and machine-executable instructions. A set of such instructions can also be called a program. The instructions, which may also be referred to as “software,” generally provide functionality by performing acts, operations, and/or methods as may be disclosed herein or understood by one skilled in the art in view of the disclosed embodiments. In some embodiments, and as a matter of convention used herein, instances of the software may be referred to as a “module” and by other similar terms. Generally, a module includes a set of instructions, to offer or fulfill a particular functionality and the processor 312 includes one or more modules. Embodiments of modules and the functionality delivered are not limited by the embodiments described in the present disclosure.

In some embodiments, the processor 312 may include the detection module 314 that further, for example, may include a Ventricular Fibrillation (VF) detector. At least one ECG signal of the ECG signals 232-242 sensed through the ECG electrodes 204-210 may be received as data by the detection module 314 from the measurement circuit 310. The data to the detection module 314 may be available as inputs or data that reflect values, or values of other signals, may be used by the VF detector to determine whether the patient 102 is experiencing VF. Detecting the VF is useful since the VF typically results in the SCA. The detection module 314 may also include a bradycardia/ventricular tachycardia/asystole detector for detecting bradycardia/ventricular tachycardia and/or asystole, and the like. The detection module 314 is also referred to as a cardiac condition detector 314. In some embodiments, a QRS detector, included in the detection module 314, may run on a single vector of the vectors 220-230. However, if multiple vectors corresponding to the vectors 220-230 are used, then a designated QRS detector may be assigned to each of the multiple vectors. Similarly, in some embodiments, a P-wave detector may run on a single vector of the vectors 220-230 depending on the actual location of the vectors 220-230. However, if multiple vectors corresponding to the vectors 220-230 are used, then a designated P-wave detector may be assigned to each of the multiple vectors.

In some embodiments, the advice module 320 may receive an output of the detection module 314 and generate advice for the one or more components of the EPD 108 regarding a subsequent course of action. The advice module 320 may provide a variety of advice based on the output of the detection module 314. In some embodiments, the advice is a shock or no shock determination to the processor 312. The shock or no shock determination may be made by executing a stored shock advisory algorithm. The shock advisory algorithm can, according to some embodiments, make a shock or no shock determination from the ECG signals 232-242 that are obtained using the ECG electrodes 204-210, and determine whether or not a shock criterion is met. The determination may be made from a rhythm analysis of the obtained ECG signals 232-242 or otherwise. For example, there may be shock decisions for the VF, VT, and the like.

In some embodiments, the advice module 320 may utilize the sensed and monitored patient parameters along with the stored shock advisory algorithm and/or the rhythm analysis of the ECG signals 232-242 for making the shock or no shock determination. Further, the advice module 320 may utilize the rhythm analysis of the ECG signals 232-242 to determine if the patient 102 needs pacing. In some embodiments, the advice module 320 may also provide inputs to the one or more output devices to indicate or notify the patient 102 regarding the impending delivery of the defibrillation shock 116 or the one or more transcutaneous pacing pulses 118, upon determining that the shock or pacing has to be provided. The advice module 320 may be capable of receiving an input from the patient 102, via the cancel switch, for aborting the delivery of the defibrillation shock 116 or the one or more transcutaneous pacing pulses 118. If the patient 102 determines an erroneous detection of a cardiac rhythm disorder, upon receiving at least a notification regarding the impending delivery of the defibrillation shock 116 or delivery of the one or more transcutaneous pacing pulses 118, then the patient 102 provides the input to abort the delivery of the defibrillation shock 116 or the one or more transcutaneous pacing pulses 118.

In some embodiments, when the one or more transcutaneous pacing pulses 118 have to be delivered and/or are being delivered to the patient 102, the medical system 100 may alert the bystanders and/or medical personnel that the medical system 100 is delivering the transcutaneous pacing. The alerts may also provide a pacing status that indicates whether intrinsic pulses were detected. In some embodiments, the pacing module 316 may control the user interface 306 to output the alerts. The alerts may help the bystander 102 to avoid interfering with the delivery of pacing while the pacing is being delivered. The alerts may also help emergency responders, such as the Emergency Medical Services (EMS) personnel, by informing the emergency responders of the pacing. Further, the EPD 108 may alert or notify the EMS personnel whether intrinsic pulses were detected. The information provided to the EMS personnel may help the EMS personnel in deciding the next course of action.

The initiation of the pacing, in some embodiments, may begin with the advice module 320 determining that the cardiac rhythm disorder is bradycardia or asystole condition. The advice module 320 may then indicate the pacing module 316 to perform the pacing. In some embodiments, after receiving the advice for the pacing, the pacing module 316 may consecutively search for an interval to deliver the one or more transcutaneous pacing pulses 118 to the patient 102, where the interval to deliver the one or more transcutaneous pacing pulses 118 is also referred to as a pacing interval. In some embodiments, the pacing module 316 is activated to provide and control the one or more transcutaneous pacing pulses 118 based on the determination of the cardiac rhythm disorder regardless of input or the advice from the advice module 320. The ECG signals 232-242 utilized for the determination of the cardiac rhythm disorder are referred to as a first set of ECG signals 232-242. In some embodiments, the first set of ECG signals may include at least one of the ECG signals 232-242.

A pacing pulse of the one or more transcutaneous pacing pulses 118, delivered to the patient 102 immediately after receiving the advice, is referred to as a first pacing pulse. The pacing module 316 may implement and/or control the ability of the EPD 108 to pace the heart 120. The ability of the pacing module 316 to pace is referred to as pacing capability. In response to detecting an arrhythmia that can be treated with pacing, for example, bradycardia, the pacing module 316 may initiate a delivery process of the one or more transcutaneous pacing pulses 118. For example, the pacing module 316 controls the power source 324, the energy output module 326, and/or the discharge circuit 328 to output the one or more transcutaneous pacing pulses 118. In embodiments that include the pacing circuit 340, the pacing module 316 may control the pacing circuit 340 and the discharge circuit 328 to output the one or more transcutaneous pacing pulses 118 to the patient 102.

In some embodiments, when the advice module 320 determines to shock the patient 102, the one or more electrical pulses are delivered at a high energy to the patient 102 by the EPD 108, through at least one of the pacing circuit 340 or the energy output module 326. Further, multiple small pulses of the one or more electrical pulses are delivered to the patient 102 in case of pacing the heart 120 of the patient 102 through the pacing circuit 340. In some embodiments, delivery pathway for shocking the patient 102 and pacing the heart 120 of the patient 102 may or may not be the same, and same or different circuit may be utilized for executing shocking and pacing functionalities without departing from the scope of the present disclosure. Delivering the one or more electrical pulses is also known as discharging, shocking the patient 102 for defibrillation, pacing, and the like. In ideal conditions, a reliable shock or no shock determination may be made by analyzing a segment of at least one ECG signal of the detected ECG signals 232-242 of the patient 102. In practice, however, the ECG signals 232-242 are often corrupted by electrical noise, which reduces the accuracy of the analyses of the ECG signals 232-242 and results in an incorrect detection of cardiac rhythm disorder, leading to a false alarm to the patient 102. Noisy ECG signals may be handled as described in U.S. patent application Ser. No. 16/037,990, filed on Jul. 17, 2018, and published as US 2019/0030351 A1, and also in U.S. patent application Ser. No. 16/038,007, filed on Jul. 17, 2018, and published as US 2019/0030352 A1, both by the same applicant and each is incorporated herein by reference.

In some embodiments, specifically the intrinsic search module 318 of the pacing module 316 may perform intrinsic search demand pacing to deliver the zero or more transcutaneous pacing pulses 118 to the patient 102. In some embodiments, the intrinsic search module 318 may initiate demand pacing at a programmed maximum pacing rate. The pacing may be continued at the maximum pacing rate for a first time interval, and then may be gradually decreased during a second interval to the minimum pacing rate to “search” for an intrinsic rhythm of the patient 102 during a third time interval or intrinsic search duration. In alternate embodiments, the intrinsic search module 318 of the pacing module 316 may perform the intrinsic search demand pacing or the fixed rate demand pacing irrespective of any fixed time duration until the battery depletion occurs. In embodiments, the processor 312 is configured to determine the number of intrinsic beats during the intrinsic search duration. The heart activity of the patient 102 exceeds the threshold when the number of intrinsic beats meets or exceeds a predetermined percentage of a total number of beats during the intrinsic search duration. Upon detecting that the heart activity of the patient 102 exceeds a threshold, the processor 312 is configured to continue causing the demand pacing function to operate at the minimum pacing rate. In accordance with embodiments, the threshold refers to a predetermined criterion for monitoring heart activity of the patient 102 during the intrinsic search duration. Specifically, it is the point at which the number of intrinsic beats of the patient 102 meets or exceeds a predefined percentage (typically between 20% and 60%) of the total number of heartbeats during the intrinsic search duration. However, if the heart activity of the patient 102 does not exceed the threshold, the processor 312 is configured to cause the demand pacing function to increase the pacing rate from the minimum pacing rate to the maximum pacing rate during a fourth time interval (or increasing pacing rate period). The intrinsic search demand pacing may continue until one or more conditions occur. By way of examples and not limitation, the one or more conditions include an alert button being activated/pressed, the medical system 100 exits being worn, or the battery of EPD 108 is depleted. In an embodiment, the alert button is same as the cancel switch described above. The detailed step-by-step process of intrinsic search demand pacing will be described in conjunction with FIG. 6 later.

The processor 312 may include additional modules, such as the configurable module 322 that, in some embodiments, is specifically coupled to an accelerometer. The combination of the configuration module 322 and the accelerometer is used to detect movement of the patient 102 that aids in determining a need for providing pacing or delivering shock to the patient 102.

The EPD 108 may also include the power source 324, which is configured to provide an electrical charge in the form of a current or one or more electrical pulses. To enable portability of the EPD 108, the power source 324, in some embodiments, may include a battery. The battery, for example, is a battery pack, which may either be rechargeable or non-rechargeable. In an example, a combination of both the rechargeable and the non-rechargeable battery packs is used. An embodiment of the power source 324 may include an alternate current (AC) power override, from where AC power may be available, an energy-storing capacitor, and the like. Appropriate components may be included to provide for charging or replacing the power source 324. In some embodiments, the power source 324 is controlled and/or monitored by the processor 312.

In some embodiments, the EPD 108 may further include the energy output module 326, which is also referred to as an energy output device 326. The energy output module 326 may be coupled to the support structure 104 either directly or via the defibrillation electrodes 112 and 114 and the respective electrode leads 110. The energy output module 326 may be coupled to receive the electrical charge provided by the power source 324. The energy output module 326 may be configured to store the electrical charge received by the power source 324. The energy output module 326 temporarily stores electrical energy in form of the electrical charge, when preparing for discharge to administer the defibrillation shock 116 or the one or more transcutaneous pacing pulses 118 to the patient 102. Hence, in some embodiments, the energy output module 326 may be referred to as an energy storage module 326. In some embodiments, the energy output module 326 may be charged from the power source 324 to the desired amount of energy as controlled by the processor 312. The energy output module 326 includes a capacitor C1, which may be a single capacitor or a system of capacitors, and the like. In some embodiments, the energy output module 326 includes a device that exhibits high power density, such as an ultracapacitor. As described above, the capacitor C1 stores the energy in the form of electrical charge, for delivering the shock, such as the defibrillation shock 116 or the one or more transcutaneous pacing pulses 118, to the patient 102. In an exemplary embodiment, the pacing circuit 340 may be powered by a separate battery or energy source to provide constant current pacing.

A decision to shock may be made responsive to the shock criterion being met, as per the above-mentioned determination. When the decision is to discharge the electrical pulses, the processor 312 may be configured to cause at least some or all of the electrical charge stored in the energy output module 326 to be discharged to the defibrillation electrodes 112 and 114 while the support structure 104 is worn by the patient 102. The discharge of the electrical pulses may include the delivery of the defibrillation shock 116 or the one or more transcutaneous pacing pulses 118 to the patient 102.

For causing the discharge, the EPD 108 may include the discharge circuit 328, also referred to as an output circuit 328. The discharge circuit 328 is coupled to the energy output module 326 and the pacing circuit 340, and in communication with the defibrillation electrodes 112 and 114. If the decision is to provide the defibrillation shock 116, the processor 312 may be configured to control the discharge circuit 328 to discharge at least some of or all of the electrical charge stored in the energy output module 326 in a desired waveform. If the decision is to merely pace, which is to deliver the one or more transcutaneous pacing pulses 118, the processor 312 may be configured to control the discharge circuit 328 to discharge at least some of the electrical charge provided by the power source 324. Since the pacing requires lesser charge and/or energy than the energy or charge for the defibrillation shock 116, in some embodiments, pacing wiring (not shown) is provided from the power source 324 to the discharge circuit 328. The pacing wiring bypasses the energy output module 326. In some embodiments, where solely the pacing is provided with no defibrillation, the energy output module 326 may not be required, or the pacing circuit 340 may be powered by a separate energy source.

A pacing current may be provided from the power source 324 via the pacing circuit 340, which may be a current source. In some embodiments, the defibrillation shock 116 is delivered using the energy output device 326, and the one or more transcutaneous pacing pulses 118 are delivered using the pacing circuit 340. In some embodiments, the pacing circuit 340 may be omitted since the EPD 108 provides the one or more transcutaneous pacing pulses 118 from the power source 324 and the energy output module 326. In some embodiments, the energy output module 326 is a current source device that provides the one or more transcutaneous pacing pulses 118. In some embodiments, the EPD 108 may include a charger (not shown) that delivers the electrical charge from the battery to the energy output module 326. In some embodiments, the charger may include a charge pump to transfer charge from the battery to the capacitor C1 of the energy output module 326. Either way, the discharging may be performed to the nodes 332 and 334 followed by the defibrillation electrodes 112 and 114 to enable delivery of the defibrillation shock 116 and/or the one or more transcutaneous pacing pulses 118 to the patient 102.

The discharge circuit 328 may include one or more switches S1. The one or more switches S1 may be made in a number of ways, such as by an H-bridge, and the like. In some embodiments, different switches S1 may be used for a discharge where the defibrillation shock 116 is caused to be delivered, than for a discharge where the weaker one or more transcutaneous pacing pulses 118 are caused to be delivered. The discharge circuit 328 may also be thus controlled via the processor 312, and/or the user interface 306. A time waveform of the discharge may be controlled by controlling the discharge circuit 328. The amount of energy of the discharge may be controlled by how much the energy output module 326 has been charged, and also by how long the discharge circuit 328 is controlled to remain open. In some embodiments, a combination of the power source 324, the energy output module 326, the discharge circuit 328, and the defibrillation or pacing port 330 coupled to the defibrillation electrodes 112 and 114 is capable of providing the defibrillation shock 116 and the one or more transcutaneous pacing pulses 118, based on the necessity, as described in U.S. Patent Application No. 63/420,523, filed on Oct. 28, 2022. In some embodiments, a combination of the power source 324, the pacing circuit 340, the discharge circuit 328, and the defibrillation or pacing port 330 coupled to the defibrillation electrodes 112 and 114 is capable of providing the one or more transcutaneous pacing pulses 118, by bypassing the energy output module 326.

The defibrillation or pacing port 330 may be a socket in the housing 302, or other equivalent structure. The defibrillation or pacing port 330 includes the nodes 332 and 334. Leads of the defibrillation electrodes 112 and 114, such as the electrode leads 110, may be plugged into the defibrillation or pacing port 330, to make electrical contact with the nodes 332 and 334, respectively. The defibrillation electrodes 112 and 114 are connected continuously to the defibrillation or pacing port 330, instead. Either way, the defibrillation or pacing port 330 may be used for guiding, via the defibrillation electrodes 112 and 114, at least some of the electrical charge that has been stored in the energy output module 326 to the patient 102. The electric charge is provided as the shock for defibrillation, pacing, and the like. In some embodiments, the defibrillation shock 116 is delivered synchronously or asynchronously. In some embodiments, the pacing module 316 provides intimation regarding the decision to deliver the one or more transcutaneous pacing pulses 118 and the user interface 306, through the one or more output devices, notifies the user, such as the bystander, that the one or more transcutaneous pacing pulses 118 are being delivered to the patient 102.

The ability of the EPD 108 to pace the heart 120 of the patient 102 may be implemented in a number of ways. The ECG electrodes 204-210 may sense at least one of the ECG signals 232-242 and the processor 312 may measure the sensed ECG signals 232-242 for delivering the zero or more transcutaneous pacing pulses 118 after the pacing interval. The pacing interval is a time duration after detection of a QRS complex in the sensed ECG signals 232-242 or after delivering the zero or more transcutaneous pacing pulses 118. The pacing may be software controlled, for example, by managing defibrillation or pacing path, or by managing the pacing circuit 340, separately, which may output the zero or more transcutaneous pacing pulses 118 to the patient 102 via the defibrillation electrodes 112 and 114.

The EPD 108 may include the communication module 338 for establishing the one or more wired or wireless communication links with other devices of other entities, such as a remote assistance center, the EMS, and the like. The communication module 338 may be similar to the communication module disclosed in FIG. 1. The communication links may be used to transfer data and commands. The data may be the patient data, event information, therapy attempted, Cardiopulmonary resuscitation (CPR) performance, system data, environmental data, and so on. The communication links, in some embodiments, may be utilized to transfer the physiological parameters data. For example, the communication module 338 may wirelessly transmit heart rate, respiratory rate, and other vital signs data daily to a server accessible over the internet, for instance as described in U.S. Patent Publication No. 2014/0043149 A1.

The physician of the patient 102 or others skilled in the art of device follow-up may directly analyze the communicated data or the communicated data may also be analyzed automatically by algorithms designed to detect a developing illness and then notify the medical personnel via text, email, phone, and the like. The communication module 338 may also include interconnected sub-components which may be deemed necessary by a person skilled in the art, for example but not limited to, an antenna, portions of the processor 312, supporting electronics, outlet for a telephone or a network cable, and the like.

The EPD 108 may further include the memory 336, which is communicatively coupled with the processor 312. The memory 336 may be implemented in a number of ways, such as, but not limited to, volatile memories, Non-Volatile Memories (NVM), Read-Only Memories (ROM), Random Access Memories (RAM), magnetic disk storage media, optical storage media, smart cards, flash memory devices, any combination thereof, and the like. The memory 336 stores one or more prompts for the user 102 if the user 102 is a local rescuer. Moreover, the memory 336 may store data including the patient data, for example, as received by the monitoring device 308. The data may be stored in memory 336 before it is transmitted out of the EPD 108, or after the data is received by the EPD 108.

The memory 336 is, thus, a non-transitory storage medium that may include programs for the processor 312, which the processor 312 may be able to read and execute. More particularly, the programs may include sets of instructions in the form of code, which the processor 312 may be able to execute upon reading. The programs may also include other information such as configuration data, profiles, scheduling, and the like, which may be acted upon by the instructions. The execution is performed by physical manipulations of physical quantities, and may result in functions, operations, processes, acts, actions, and/or methods to be performed. In some embodiments, the processor 312 is configured to cause other devices or components or blocks to perform functions, operations, processes, acts, actions and/or methods mentioned above. The programs may be operational for the inherent needs of the processor 312 and may also include protocols and to assist the advice module 320 in decision-making.

The non-transitory computer-readable storage medium is encoded or configured to store computer program instructions, which are pacing pulse delivery and verification instructions, defined by modules, for example, 306, 308, 310, 314, 316, 318, 320, 322, 338, 340, and the like, which when executed by a computing device, such as the EPD 108 or the processor 312, cause the computing device to perform operations for delivering and verifying the one or more transcutaneous pacing pulses 118 to the patient 102.

The operations include receiving the first set of the ECG signals 232-242 of the patient 102 via one or more of the ECG electrodes 204-210. The operations further include determining, by the cardiac condition detector 314, if the first set of ECG signals 232-242 are indicative of a cardiac condition or cardiac rhythm disorders treatable by pacing. If the cardiac condition is determined to be treatable by pacing, the demand pacing function operable by the processor 312 of EPD 108 is enabled. However, the pacing pulses may not necessarily be delivered. The one or more pacing pulses 118 are only delivered when the intrinsic heart rate of the patient 102 is less than the pacing rate of the EPD 108. As explained above, the EPD 108 is configured to provide demand pacing to the patient 102 when necessary. Specifically, the processor 312 is configured to operate the EPD 108 according to the demand pacing function which includes a fixed pacing demand mode or an intrinsic search pacing demand mode. The demand pacing function is operable to initiate delivery of the one or more transcutaneous pacing pulses 118 when intrinsic heart rate of the patient 102 is not detected, or when the intrinsic heart rate of the patient 102 falls below the set pacing rate of the EPD 108. This ensures that the one or more transcutaneous pacing pulses 118 are delivered only when required, allowing the patient's intrinsic heart activity to be preserved as much as possible.

FIG. 4A illustrates a first pacing scenario 400A for intrinsic search demand pacing depicting the occurrence of various transitions during the intrinsic search demand pacing, in accordance with an embodiment of the present disclosure. FIG. 4A is described in conjunction with previous figures.

In particular, the first pacing scenario 400A depicts a state in which intrinsic heart activity of the patient 102 exceeds a threshold during a third time interval (or intrinsic search duration). In embodiments, the pacing module 316 present in the EPD 108 paces the heart 120 of the patient 102 when the intrinsic heart activity of the patient 102 is less than the set pacing rate of the EPD 108. As depicted in FIG. 4A, the first pacing scenario 400A includes a maximum pacing rate 402, a decreasing pacing rate period 404, and a minimum pacing rate 406. FIG. 4A is described assuming an exemplary value of the maximum pacing rate 402 as 80 bpm that corresponds to an interval of 0.75 seconds, the minimum pacing rate 406 as 40 bpm that corresponds to an interval of 1.5 seconds, and the intrinsic heart rate of the patient 102 as 60 bpm that corresponds to an interval of 1 second.

In embodiments, the cardiac condition detector 314 of the EPD 108 or the medical system 100 may detect the condition of the patient 102 (such as bradycardia, tachycardia, or asystole) based on received ECG signals 232-242 of the patient 102 via one or more of the ECG electrodes 204-210. Based on the detected condition, the medical system 100 may determine whether the patient 102 is in need of pacing. In embodiments, the pacing may be needed when the patient 102 experiences no intrinsic beats. In general, demand pacing is the mode of pacing that allows paced beats to be delivered on a beat-to-beat basis to fill in gaps in the intrinsic rhythm. In embodiments, the medical system 100 is configured to provide demand pacing to the patient 102 to treat the detected heart condition. In order to provide demand pacing, the processor 312 is configured to operate the medical system 100 according to the demand pacing function. The demand pacing function is configured to initiate delivery of the zero or more transcutaneous pacing pulses 118. Specifically, the demand pacing function is operable to deliver one or more transcutaneous pacing pulses 118 when an intrinsic beat (or patient heartbeat) is not detected at a given rate. For example, when the rate is 80 bpm, there are 80 windows to deliver pacing pulses in a single minute. For each window where an intrinsic beat is not detected, the demand pacing function is configured to cause a pacing pulse to be delivered. Further, the demand pacing function may be operated either in a fixed pacing demand mode or an intrinsic search pacing demand mode.

During the intrinsic search pacing demand mode, the medical system 100 sets the pacing rate to the maximum pacing rate 402 for a first time interval, as depicted in FIG. 4A. The first time interval may be a fixed or a non-fixed time interval. When fixed, the first time interval may correspond to the time set by the clinician or technician or Patient Service Representative (PSR) based on the need of pacing at the maximum pacing rate 402. Specifically, the first time interval corresponds to a time duration during which the medical system 100 (e.g., the EPD 108) stays at the maximum pacing rate 402. In an exemplary embodiment, a typical value for the first time interval may be 5 minutes. Further, the pacing interval between the consecutive pacing pulses during the first time interval may be 0.75 seconds, as shown in FIG. 4A. As mentioned above, the maximum pacing rate 402 may be set at 80 bpm for the first time interval. In embodiments, the first time interval may also be a fixed time, not changeable by any user. In the current example, with the maximum pacing rate 402 set at 80 bpm with the first time interval of 5 minutes and the intrinsic heart rate of 60 bpm with time interval of 1 second, the one or more transcutaneous pacing pulses 118 may be continually produced. However, the amount of the one or more transcutaneous pacing pulses 118 that actually get delivered during the first time interval is driven by the number of intrinsic beats that occur. Specifically, the detection of intrinsic beats at a set rate causes paced beats to be inhibited. The pacing beats will only be delivered in the absence of intrinsic beats within a pacing window defined by the demand pacing function.

After the expiration of the first time interval, the medical system 100 is configured to gradually decrease the pacing rate from the maximum pacing rate 402 (such as 80 bpm) to the minimum pacing rate 406 during a second time interval 404 (or decreasing pacing rate period 404). The second time interval 404 may be a fixed or a non-fixed time interval. In embodiments, a typical value for the second time interval 404 may be 10 seconds. Once the minimum pacing rate 406 is achieved, the medical system 100 enters the third time interval from the second time interval 404. In embodiments, the third time interval (or the intrinsic search duration) may either be a fixed time interval or a fixed number of beats (including both paced and intrinsic beats). In an example, the fixed number of beats to determine whether the intrinsic beats of the patient 102 exceeds the threshold may be five. Further, in a non-limiting example, the minimum pacing rate 406 is achieved anywhere between 20 bpm to 60 bpm.

During the third time interval, the medical system 100 enters the intrinsic search duration to determine whether the intrinsic heart activity of the patient 102 exceeds the threshold or not. The threshold is the point at which the number of intrinsic beats of the patient 102 meets or exceeds a predefined percentage (typically between 20% and 60%) of the total number of heartbeats during the intrinsic search duration. As depicted in FIG. 4A, when the intrinsic heart activity of the patient 102 exceeds the threshold, the medical system 100 continues operating at the minimum pacing rate 406. Further, the medical system 100 inhibits pacing when the value of the intrinsic heart rate (e.g., 60 bpm) of the patient 102 exceeds the minimum pacing rate 406 (e.g., 40 bpm) set by the medical system 100 (e.g., the pacemaker). The threshold criteria may be different for different embodiments.

At the minimum pacing rate 406, the medical system 100 monitors if the heart 120 of the patient 102 is generating more intrinsic beats than the set pacing rate. If the heart 120 of the patient 102 is generating more intrinsic beats, the medical system 100 stays at the minimum pacing rate 406. As will be described in FIG. 4B in detail, if the intrinsic heart activity of the patient 102 does not exceed the threshold (i.e., insufficient intrinsic beats are detected), the medical system 100 increases the pacing rate back to the maximum pacing rate 402 over a fourth time interval 408 (or the increasing pacing rate period 108), which may be fixed or non-fixed (e.g., 5 seconds).

FIG. 4B illustrates a second pacing scenario 400B for intrinsic search demand pacing depicting the occurrence of various transitions during the intrinsic search demand pacing, according to an embodiment of the present disclosure. In particular, the second pacing scenario 400B depicts a state in which the intrinsic heart activity of the patient 102 does not exceed the threshold during the third time interval. FIG. 4B is described conjunction with previous figures. As depicted in FIG. 4B, the second pacing scenario 400B includes the maximum pacing rate 402, the decreasing pacing rate period 404, the minimum pacing rate 406, and the increasing pacing rate period 408. FIG. 4B is described assuming an exemplary value of the maximum pacing rate 402 as 80 bpm that corresponds to an interval of 0.75 seconds, the minimum pacing rate 406 as 40 bpm that corresponds to an interval of 1.5 seconds, and the intrinsic heart rate of the patient 102 as 30 bpm that corresponds to an interval of 2 seconds.

In accordance with the embodiments, the pacing is performed throughout the second pacing scenario 400B since the intrinsic heart activity of the patient 102 does not exceed the threshold during the third time interval. In contrast to the first pacing scenario 400A, the intrinsic beats of the patient 102 remain less than the pacing rate during the third time interval which leads to the second pacing scenario 400B.

During the intrinsic search pacing demand mode, the medical system 100 sets the pacing rate to the maximum pacing rate 402 for the first time interval, as depicted in FIG. 4B. With the intrinsic heart rate of 30 bpm and the maximum pacing rate 402 set at 80 bpm, the one or more transcutaneous pacing pulses 118 are continually produced. However, the amount of the one or more transcutaneous pacing pulses 118 that actually get delivered during the first time interval is driven by the number of intrinsic beats that occur. Specifically, the detection of intrinsic beats at a set rate causes paced beats to be inhibited. The pacing beats will only be delivered in the absence of intrinsic beats within a pacing window defined by the demand pacing function.

After the expiration of the first time interval, the processor 312 is configured to cause the demand pacing function to enter the second time interval 404 or the decreasing pacing rate period 404, which further leads to entering the third time interval. During the decreasing pacing rate period 404, the medical system 100 decreases the pacing rate from the maximum pacing rate 402 to the minimum pacing rate 406.

As depicted in FIG. 4B, during the intrinsic search duration or the third time interval, the intrinsic heart rate (e.g., 30 bpm) of the patient 102 is less than the minimum pacing rate 406 (e.g., 40 bpm) i.e., the intrinsic heart activity does not exceed the threshold. As a result, the one or more transcutaneous pacing pulses 118 are delivered to the patient 102 and no intrinsic heart activity occurs. The medical system 100 subsequently enters the increasing pacing rate period 408 (or the fourth time interval 408) as a result of lack of sufficient intrinsic heart activity in the third time interval, which further leads to entering the first time interval. During the increasing pacing rate period 408, the medical system 100 increases the pacing rate back from the minimum pacing rate 406 to the maximum pacing rate 402 over a set period (e.g., 5 seconds). In an example, in the increasing pacing rate period 408, with an intrinsic heart rate of 30 bpm and the maximum pacing rate 402 of 80 bpm, the one or more transcutaneous pacing pulses 118 are continually produced at the maximum pacing rate 402. Therefore, FIG. 4B depicts the scenario when sufficient intrinsic beats are not detected for the patient 102, and as a result, the threshold is not exceeded when compared with the set pacing rate.

It shall be noted that the values of time intervals, the maximum pacing rate 402, the decreasing pacing rate period 404, the minimum pacing rate 406, and the increasing pacing rate period 408 in FIG. 4A and FIG. 4B may vary depending upon the medical condition of the patient 102. Further, such values may dynamically change (i.e., non-fixed) while attempting to detect the intrinsic activity of the patient 102.

To summarize, in the present embodiments describing intrinsic search demand pacing, the pacing rate may oscillate between a minimum pacing rate and a maximum pacing rate, where the pacing rate may stay at the minimum pacing rate (e.g., 406) for longer time duration if sufficient intrinsic heart activity of the patient 102 is detected, and the pacing rate will continue to probe down from the maximum pacing rate (e.g., 402) to the minimum pacing rate (e.g., 406) to search for the intrinsic heart activity of the patient 102.

In alternate embodiments, the processor 312 may cause the demand pacing function to operate in demand mode, irrespective of any fixed time duration, until the battery depletion occurs.

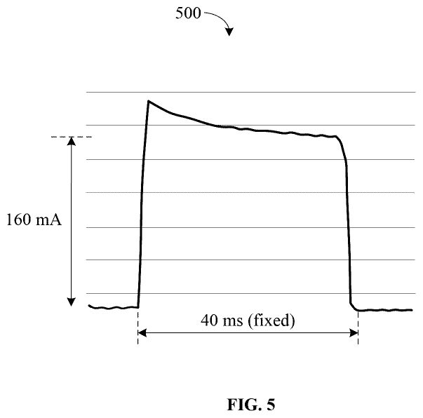

FIG. 5 illustrates a pacing pulse waveform 500 for pacing, according to an embodiment of the present disclosure. FIG. 5 is described in conjunction with previous figures. In an exemplary embodiment, the cardiac condition detector 314 of the EPD 108 or the medical system 100 detects the medical condition of the patient 102 using ECG signals 232-242. Based on the detected medical condition of the patient 102, if pacing is needed, the clinician may select either the intrinsic search pacing demand mode or the fixed pacing demand mode of the demand pacing function.

In either of the demand pacing modes described above, the zero or more transcutaneous pacing pulses 118 may be delivered on a beat-to-beat basis to fill in the gaps in the intrinsic rhythm of the patient 102 as described above. In FIG. 5, a pacing pulse waveform 500 that is common for fixed rate demand pacing and intrinsic search demand pacing is exemplified. The exemplary pacing current may be set to 160 mA for a duration of 40 ms per pulse.

FIG. 6 illustrates a flowchart 600 for the fixed rate demand pacing and intrinsic search demand pacing, according to an embodiment of the present disclosure. FIG. 6 is described in conjunction with previous figures. In embodiments, the demand pacing function of the medical system 100 (e.g., pacemaker or the EPD 108) is set to either the fixed pacing demand mode or the intrinsic search pacing demand mode. The setting of the demand pacing function may be performed by the clinician or technician, rather than the patient 102, or autonomously by the medical system 100. In other words, the setting of the demand pacing function may not be dynamically changeable and once set, the medical system 100 can only provide one type of pacing (either the fixed rate demand pacing or the intrinsic search demand pacing). In other embodiments, the setting of the demand pacing function may be dynamically changeable based on the need of the patient 102.

Referring to FIG. 6, at step 602, the medical system 100 (e.g., the EPD 108) provides options to the clinician (or medical professional or technician) for managing the rate of the EPD 108 by providing a demand pacing function. The options may be selecting either the fixed pacing demand mode or the intrinsic search pacing demand mode. The clinician may set one of the demand pacing modes, based on the need of the patient 102. The need of the patient 102 is decided based on the detected condition (such as bradycardia or asystole), which is determined by the ECG signals 232-242 via the plurality of ECG electrodes 204-210.

In response to the clinician selecting the mode as fixed pacing demand mode, the medical system 100, at step 604, initiates the fixed rate demand pacing and delivers the zero or more transcutaneous pacing pulses 118 to the patient 102 via the at least one therapy electrode 112, 114 at a fixed pacing rate. The one or more transcutaneous pulses 118 are delivered when the patient's intrinsic rate is less than the fixed, programmed rate. The fixed rate demand pacing will continue until one or more conditions occur. The one or more conditions, without limitation and by way of examples, include an alert button being activated/pressed, the medical system 100 exits being worn by the patient 102, or the battery of the EPD 108 is depleted. In embodiments, the maximum pacing rate 402 and the minimum pacing rate 406 may be equal to provide the fixed pacing rate.

In response to the clinician setting the mode as intrinsic search pacing demand mode, the medical system 100, at step 606, initiates the intrinsic search demand pacing using the intrinsic search module 318. In an exemplary embodiment, the intrinsic search module 318 is configured to search for the intrinsic rhythm of the patient 102. Specifically, the intrinsic search module 318 may cause the pacing rate to be set to a maximum pacing rate for a first time interval. In other words, the processor 312 of the EPD 108, comprising the intrinsic search module 318, is configured to cause the demand pacing function to operate at the maximum pacing rate 402 for the first time interval. In a non-limiting example, the maximum pacing rate 402 may be 80 bpm, and the first time interval may be 5 minutes. The values of the maximum pacing rate 402 and/or the first time interval can be programmable in some embodiments, without departing from the scope of the present disclosure.

Further, after expiration of the first time interval, the intrinsic search module 318, at step 608, is configured to gradually decrease the pacing rate from the maximum pacing rate 402 to the minimum pacing rate 406 over a decreasing pacing rate period 404 or the second time interval. In a non-limiting example, the minimum pacing rate 406 may be 40 bpm, and the second time interval 404 may be 10 seconds. The values of the minimum pacing rate 406 and/or the second time interval 404 can be programmable in some embodiments, without departing from the scope of the present disclosure.

Further, the processor 312 of the EPD 108 is configured to operate the demand pacing function at the minimum pacing rate 406 for a third time interval. Subsequently, once the minimum pacing rate 406 is achieved, the patient 102 is monitored for a number of intrinsic beats during an intrinsic search duration (or the third time interval), at step 610. Specifically, each beat is monitored during the intrinsic search duration to categorize the beat as a sensed event (i.e., an intrinsic heartbeat) or a paced event (i.e., paced beat). The intrinsic search module 318, at step 610, is configured to determine, via one or more of the ECG signals 232-242, whether a heart activity of the patient 102 exceeds a threshold during the intrinsic search duration. The heart activity of the patient 102 may include zero or more intrinsic beats and/or zero or more paced beats during the intrinsic search duration. The processor 312 is configured to determine the number of intrinsic beats during the intrinsic search duration. As a non-limiting example, the threshold may correspond to detection of about 20 percent to about 60 percent of a total number of beats in the intrinsic search duration. As another non-limiting example, the threshold may correspond to detection of two beats over a total of five beats. However, the threshold may not be limited to aforementioned value or a range and may extend to different values or ranges based on the monitoring of heart activity during the course of intrinsic search demand pacing.

If the heart activity of the patient 102 exceeds the threshold, the intrinsic search module 318 causes the pacing rate to stay at the minimum pacing rate 406, at step 610. Therefore, the intrinsic search module 318 is configured to perform the intrinsic search for the intrinsic rhythm at the minimum pacing rate 406. Accordingly, based on the threshold, the intrinsic search module 318 is configured to determine whether sufficient intrinsic beats are detected and accordingly continues to cause the EPD 108 to stay at the minimum pacing rate 406, thereby favoring the intrinsic beats over the paced beats.

If the heart activity of the patient 102 does not exceed the threshold, the intrinsic search module 318 is configured to gradually increase the pacing rate from the minimum pacing rate 406 to the maximum pacing rate 402 during the increasing pacing rate period 408 (or the fourth time interval 408), at step 612. In other words, if the intrinsic heat rate of the patient 102 is less than the set pacing rate of the EPD 108, the pacing rate is increased during the increasing pacing rate period 408 or the fourth time interval 408. In a non-limiting example, the fourth time interval 408 may be 5 seconds. The pacing will continue at step 612 until the maximum pacing rate 402 is achieved.

In some embodiments, the assessment of whether there is sufficient heart activity to stay in the minimum pacing rate 406 or whether there is insufficient heart activity with a need to return to the maximum pacing rate 402 may be performed over a period of time such as but not limited to 15 seconds. In other embodiments, the assessment may be performed for a predetermined number of beats and pulses such as but not limited to five beats and pulses.

Subsequently, the intrinsic search module 318 is configured to maintain the pacing rate at the maximum pacing rate 402 at step 606 and then iteratively perform the above-described steps 608, 610, and 612 until the heart activity of the patient 102 exceeds the threshold to detect the intrinsic beats. As previously stated, intrinsic beats are believed to be better for the heart 120 of the patient 102. In accordance with embodiments of the present disclosure, it is believed that pacing at a high rate may prevent intrinsic beats from occurring or reduce the occurrence of intrinsic beats. Accordingly, the intrinsic search demand pacing described in these embodiments can provide more opportunities for the heart 120 of the patient 102 to generate intrinsic beats.

In the embodiments presented in FIG. 6, the values of pacing rates and times/durations can be different and may be extended to other values or ranges without limitation. To summarize, the fixed pacing demand mode or the intrinsic search pacing demand mode is programmed, which results in a fixed pacing rate or intrinsic search pacing rate (i.e., maximum to minimum). In other words, demand pacing is provided either by pacing at a fixed rate or automatically varying/reducing the fixed rate occasionally to search for intrinsic heart rates of the patient 102.

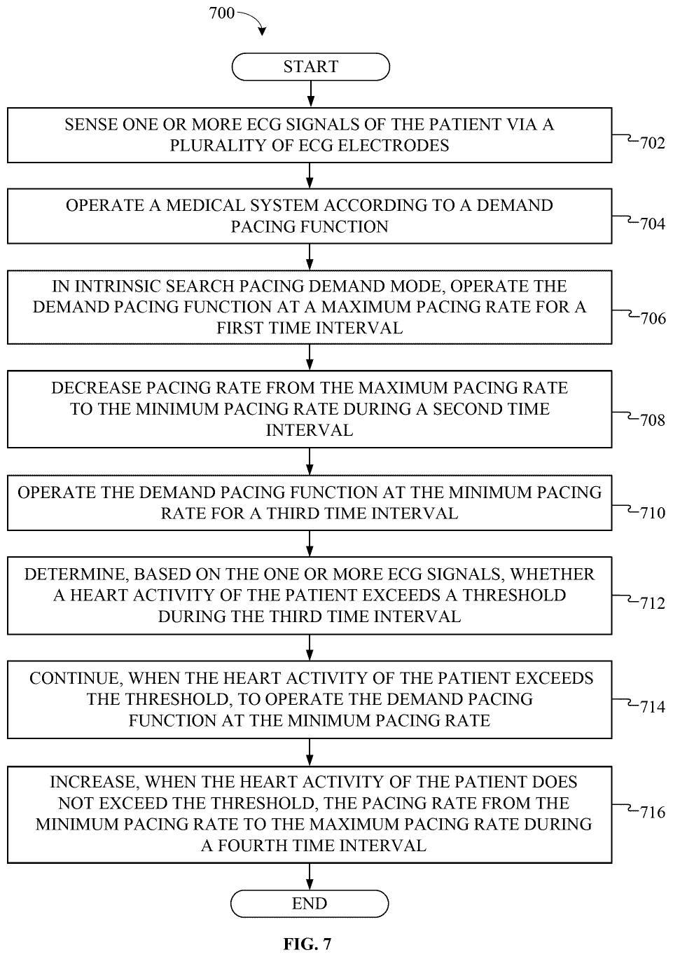

FIG. 7 illustrates an example method 700 for managing a pacing function in a medical system, according to an embodiment of the present disclosure. Method 700 will be explained in conjunction with the previous figures. Although the example utilized for the method 700 depicts a particular sequence of operations, the sequence may be altered without departing from the scope of the present disclosure. For example, some of the operations depicted may be performed in parallel or in a different sequence that does not materially affect the function of the method 700. In other examples, different components of an example apparatus or system that implements the method 700 may perform functions at substantially the same time or in a specific sequence.

The method 700 begins at block 702 by sensing the one or more ECG signals 232-242 of the patient 102 via the plurality of ECG electrodes 204-210. In response to sensing the one or more ECG signals 232-242, the medical system 100 determines whether the patient 102 is in need of pacing. The need of pacing is based on a condition of the patient 102 (such as but not limited to bradycardia or asystole) which is determined based on the sensed one or more ECG signals 232-242.

Upon determining that the patient 102 is in need of pacing, the method 700 includes operating the medical system 100 according to the demand pacing function at block 704. The medical system 100 includes the plurality of ECG electrodes 204-210 and the at least one therapy electrode 112, 114. The at least one therapy electrode 112, 114 may be positioned external to the patient 102. In some embodiments, the medical system 100 may receive a user input to operate the pacing function in the fixed pacing demand mode or the intrinsic search pacing demand mode. A selection of one of the demand pacing modes may be received as the user input by the medical system 100. The user input may be received via the communication module 338. However, the user input for selecting a demand pacing mode is optional, and the medical system 100 may be configured to select a demand pacing mode automatically without manual intervention. The demand pacing function is operable to initiate the zero or more transcutaneous pacing pulses 118 to be delivered by the at least one therapy electrode 112, 114, for example, via the pacing circuit 340 and/or the defibrillation or pacing port 330.

After the selection of the mode as the intrinsic search pacing demand mode, the method 700 includes operating the demand pacing function at the maximum pacing rate 402 for a first time interval, at block 706. The first time interval is a time interval (e.g., 5 minutes) which may be preprogrammed or controlled by the medical system 100. Specifically, the processor 312 of the medical system 100, in communication with the plurality of ECG electrodes 204-210 and the at least one therapy electrode 112, 114, causes the medical system 100 to be set to the maximum pacing rate 402 for the first time interval, which may result in zero or more pacing pulses 118 to be delivered to the patient 102. As an example, the maximum pacing rate 402 may have a default value of 80 bpm. As another example, the maximum pacing rate 402 may be selected from a range of 30 bpm to 100 bpm based on the medical condition of the patient 102. However, the disclosure is not limited to aforementioned value and range, and may include other values and ranges without limitation.

Further, the method 700 includes decreasing the pacing rate from the maximum pacing rate 402 to the minimum pacing rate 406 during a second time interval 404 or the decreasing pacing rate period 404, at block 708. Furthermore, the method 700 includes operating the demand pacing function at the minimum pacing rate 404 for an intrinsic search duration, at block 710. The heart activity of the patient 102 comprises zero or more intrinsic beats and/or zero or more paced beats during the intrinsic search duration. In a non-limiting example, the pacing rate may be reduced from 80 bpm to 40 bpm over a period of 10 seconds.

Further, the method 700 includes determining, based on the one or more ECG signals 232-242, whether the heart activity of the patient 102 exceeds a specific criteria or a threshold during the intrinsic search duration, at block 712. The specific criteria or the threshold may be configured for determining sufficiency of heart activity or for qualifying an event as the sensed event. The criteria or threshold may include sensing a specific number of intrinsic beats out of a total number of beats in the intrinsic search duration. It shall be noted that criteria or thresholds may be different for different embodiments and may be programmed or configured on a case-to-case basis (i.e., different for different patients and/or associated medical conditions).

In an exemplary embodiment, the heart activity of the patient 102 exceeds the threshold during the intrinsic search duration if the number of intrinsic beats in the intrinsic search duration exceeds a predetermined percentage of a total number of beats during the intrinsic search duration. When the heart activity of the patient 102 exceeds the threshold, it serves as an indication that the intrinsic heart rate of the patient 102 is more than the set pacing rate of the EPD 108 (or the medical system 100).

The method 700 further includes continuing, when the heart activity of the patient 102 exceeds the threshold during the intrinsic search duration, to operate the demand pacing function at the minimum pacing rate 404, at block 714. Accordingly, the intrinsic rhythm of the patient 102 is sensed by the medical system 100, causing the medical system 100 to continue operating the demand pacing function at the minimum pacing rate 406 and favor intrinsic beats over paced beats. In accordance with the above embodiments, the demand pacing is termed as rescue pacing as the medical system 100 first detects a significant pause/severe bradycardia on the order of 10-60 seconds, then alarms the patient 102 to give them a chance to press the alert button to avoid pacing therapy.

Further, the method 700 includes operating, when the heart activity of the patient 102 does not exceed the threshold, the demand pacing function at the maximum pacing rate 402 during an increasing pacing rate period 408, at block 716. As a non-limiting example, the pacing rate may be gradually increased from 40 bpm to 80 bpm over 5 seconds. After the maximum pacing rate 402 is achieved, the medical system 100 may cause the one or more transcutaneous pacing pulses 118 to be delivered at the first time interval. Subsequently, the process is repeated to detect the intrinsic heart rate or search for the intrinsic rhythm of the patient 102. In other embodiments, the medical system 100 may operate the demand pacing function at a fixed pacing rate, as previously described with reference to previous figures.

Therefore, in various embodiments described, the medical system 100 provides an improvement over the existing WCD systems by operating a demand pacing function with an ability to provide configurable pacing rate. The medical system 100 provides therapy or treatment to patients using pacing in response to detecting arrhythmia(s) that might be treated with pacing. The medical system 100 can swiftly intervene and deliver life-saving shocks or treatments to rescue the heart 120 of the patient 102 from arrhythmias or cardiac arrest. Hence, the medical system 100 preserves the patient's well-being and potentially prevents fatal outcomes. Advantageously, the medical system 100 provides customized therapy by utilizing the fixed pacing demand mode and intrinsic search pacing demand mode of the demand pacing function, in case of abnormal cardiac activity.

Other embodiments include combinations and sub-combinations of features described or shown in the drawings herein, including for example, embodiments that are equivalent to providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, feature or features can refer to the structures and/or functions of an apparatus, article of manufacture or system, and/or the steps, acts, or modalities of a method.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A medical system, comprising:
a support structure configured to be worn by a patient;
a plurality of electrocardiogram (ECG) electrodes to sense one or more ECG signals of the patient;
at least one therapy electrode engaged to the support structure and in communication with an energy source, the at least one therapy electrode being configured to deliver one or more pacing pulses to the patient; and
a processor in communication with the plurality of ECG electrodes and the at least one therapy electrode, the processor being configured to operate the medical system according to a demand pacing function;
wherein the demand pacing function is configured to initiate delivery of zero or more pacing pulses;
wherein the demand pacing function comprises an intrinsic search pacing demand mode; and
wherein, in the intrinsic search pacing demand mode, the processor is further configured to:
cause the demand pacing function to operate at a maximum pacing rate for a first time interval;
cause the demand pacing function to operate at a minimum pacing rate for an intrinsic search duration; and
determine, via the one or more ECG signals, whether a heart activity of the patient exceeds a threshold during the intrinsic search duration.

2. The medical system of claim 1, wherein the heart activity of the patient comprises zero or more intrinsic beats and/or zero or more paced beats during the intrinsic search duration.

3. The medical system of claim 2, wherein the processor is further configured to determine a number of intrinsic beats that occurred during the intrinsic search duration.

4. The medical system of claim 3, wherein the heart activity of the patient exceeds the threshold when the number of intrinsic beats meets or exceeds a predetermined percentage of a total number of beats during the intrinsic search duration.

5. The medical system of claim 1, wherein when the heart activity of the patient exceeds the threshold, the processor is configured to continue causing the demand pacing function to operate at the minimum pacing rate.

6. The medical system of claim 1, wherein the processor is further configured to decrease pacing rate from the maximum pacing rate to the minimum pacing rate during a decreasing pacing rate period.

7. The medical system of claim 1, wherein when the heart activity of the patient does not exceed the threshold, the processor is configured to cause the demand pacing function to operate at the maximum pacing rate for the first time interval.

8. The medical system of claim 1, wherein the processor is further configured to increase pacing rate from a minimum pacing rate to the maximum pacing rate during an increasing pacing rate period.

9. The medical system of claim 1, wherein the demand pacing function further comprises a fixed pacing demand mode, and wherein, in the fixed pacing demand mode, the processor is configured to cause the demand pacing function to operate at a fixed pacing rate.

10. A medical system, comprising:
a support structure configured to be worn by a patient;
a plurality of electrocardiogram (ECG) electrodes to sense one or more ECG signals of the patient;
at least one therapy electrode engaged to the support structure and in communication with an energy source, the at least one therapy electrode being configured to deliver one or more pacing pulses to the patient; and
a processor in communication with the plurality of ECG electrodes and the at least one therapy electrode, the processor being configured to:
operate the medical system according to a demand pacing function comprising a fixed pacing demand mode and an intrinsic search pacing demand mode; and
receive an input for the fixed pacing demand mode or the intrinsic search pacing demand mode;
wherein the demand pacing function is operable to initiate delivery of zero or more pacing pulses;
wherein, in the intrinsic search pacing demand mode, the processor is further configured to:
cause the demand pacing function to operate at a maximum pacing rate for a first time interval;
cause the demand pacing function to operate at a minimum pacing rate for an intrinsic search duration; and
determine, via the one or more ECG signals, whether a heart activity of the patient exceeds a threshold during the intrinsic search duration; and
wherein, in the fixed pacing demand mode, the processor is further configured to operate the demand pacing function at a fixed pacing rate.

11. The medical system of claim 10, wherein the heart activity of the patient comprises zero or more intrinsic beats and/or zero or more paced beats during the intrinsic search duration.

12. The medical system of claim 11, wherein the processor is further configured to determine a number of intrinsic beats during the intrinsic search duration.

13. The medical system of claim 12, wherein the heart activity of the patient exceeds the threshold when the number of intrinsic beats meets or exceeds a predetermined percentage of a total number of beats during the intrinsic search duration.

14. The medical system of claim 10, wherein when the heart activity of the patient exceeds the threshold, the processor is configured to continue operating the demand pacing function at the minimum pacing rate.

15. The medical system of claim 10, wherein the processor is further configured to decrease pacing rate from the maximum pacing rate to the minimum pacing rate during a decreasing pacing rate period.

16. The medical system of claim 10, wherein when the heart activity of the patient does not exceed the threshold, the processor is configured to operate the demand pacing function at the maximum pacing rate for the first time interval.

17. The medical system of claim 10, wherein the processor is further configured to increase pacing rate from a minimum pacing rate to the maximum pacing rate during an increasing pacing rate period.

18. A medical system, comprising:
a support structure configured to be worn by a patient;
a plurality of electrocardiogram (ECG) electrodes to sense one or more ECG signals of the patient;
at least one therapy electrode engaged to the support structure and in communication with an energy source and configured to deliver one or more pacing pulses to the patient; and
a processor, in communication with the plurality of ECG electrodes and the at least one therapy electrode, the processor being configured to operate the medical system according to a demand pacing function;
wherein the demand pacing function is configured to initiate delivery of zero or more pacing pulses;
wherein the demand pacing function comprises a fixed pacing demand mode; and
wherein, in the fixed pacing demand mode, the processor is configured to cause the demand pacing function to operate at a fixed pacing rate.

19. The medical system of claim 18, wherein the at least one therapy electrode is positioned external to the patient.

20. The medical system of claim 18, wherein the one or more pacing pulses are transcutaneous pacing pulses.

21. A method for managing a demand pacing function in a medical system, the method comprising:
sensing one or more electrocardiogram (ECG) signals of a patient via a plurality of ECG electrodes; and
operating the medical system according to a demand pacing function, the medical system comprising the plurality of ECG electrodes and at least one therapy electrode;
wherein the demand pacing function is operable to initiate zero or more pacing pulses to be delivered by the at least one therapy electrode;
wherein the demand pacing function comprises an intrinsic search pacing demand mode;
wherein, in the intrinsic search pacing demand mode, the method further comprises:
operating the demand pacing function at a maximum pacing rate for a first time interval;
operating the demand pacing function at a minimum pacing rate for an intrinsic search duration; and
determining, based on the one or more ECG signals, whether a heart activity of the patient exceeds a threshold during the intrinsic search duration.

22. The method of claim 21, wherein the demand pacing function further comprises a fixed mode, and wherein, in the fixed mode, the method further comprises operating the demand pacing function at a fixed pacing rate.

23. The method of claim 21, wherein the heart activity of the patient comprises zero or more intrinsic beats and/or zero or more paced beats during the intrinsic search duration, wherein the method further comprises determining a number of intrinsic beats during the intrinsic search duration, and wherein the heart activity of the patient exceeds the threshold when the number of intrinsic beats in the intrinsic search duration exceeds a predetermined percentage of a total number of beats during the intrinsic search duration.

24. The method of claim 23, wherein when the heart activity of the patient exceeds the threshold, the method further comprises continuing to operate the demand pacing function at the minimum pacing rate.

25. The method of claim 23, wherein when the heart activity of the patient does not exceed the threshold, the method further comprises operating the demand pacing function at the maximum pacing rate for the first time interval.

26. A method for managing a demand pacing function in a medical system, the method comprising:
sensing one or more electrocardiogram (ECG) signals of a patient via a plurality of ECG electrodes;
operating the medical system according to a demand pacing function, the medical system comprising the plurality of ECG electrodes and at least one therapy electrode; and
receiving a user input to operate the demand pacing function in a fixed pacing demand mode or an intrinsic search pacing demand mode;
wherein the demand pacing function is operable to initiate zero or more pacing pulses to be delivered by the at least one therapy electrode;
wherein, in the intrinsic search pacing demand mode, the method further comprises:
operating the demand pacing function at a maximum pacing rate for a first time interval;
operating the demand pacing function at a minimum pacing rate for an intrinsic search duration; and
determining, via the one or more ECG signals, whether a heart activity of the patient exceeds a threshold during the intrinsic search duration; and
wherein, in the fixed pacing demand mode, the method further comprises operating the demand pacing function at a fixed pacing rate.

27. The method of claim 26, wherein the heart activity of the patient comprises zero or more intrinsic beats and/or zero or more paced beats during the intrinsic search duration, wherein the method further comprises determining a number of intrinsic beats during the intrinsic search duration, and wherein the heart activity of the patient exceeds the threshold when the number of intrinsic beats in the intrinsic search duration exceeds a predetermined percentage of a total number of beats during the intrinsic search duration.

28. The method of claim 26, wherein when the heart activity of the patient exceeds the threshold, the method further comprises continuing to operate the demand pacing function at the minimum pacing rate.

29. A method for managing a demand pacing function in a medical system, the method comprising:
sensing one of more electrocardiogram (ECG) signals of a patient via a plurality of ECG electrodes; and
operating the medical system according to a demand pacing function, the medical system comprising the plurality of ECG electrodes and at least one therapy electrode;
wherein the demand pacing function is configured to initiate zero or more pacing pulses to be delivered by the at least one therapy electrode;
wherein the demand pacing function comprises a fixed pacing demand mode; and
wherein, in the fixed pacing demand mode, the method further comprises operating the demand pacing function at a fixed pacing rate.

30. The method of claim 29, wherein the at least one therapy electrode is positioned external to the patient.

31. The method of claim 29, wherein pacing pulses being delivered are transcutaneous pacing pulses.
